(19) United States
(12) Patent Application Publication
(10) Pub. No.: US 2010/0028916 A1
(43) Pub. Date: Feb. 4, 2010

(54) Method for the detection of enzymatic reactions

(75) Inventors: Abraham Ambar, Grenzach 1 (DE); Benjamin Badri, Zurich (CH)

Correspondence Address: CLARK & ELBING LLP, 101 FEDERAL STREET, BOSTON, MA, 02110, US

(73) Assignee: Bio Pur AG, Bubendorf (CH)

(21) Appl. No.: 12/308,140

(22) PCT Filed: Jun. 8, 2007
(86) PCT No.: PCT/EP2007/005078

§ 371 Date: Aug. 25, 2009

(30) Foreign Application Priority Data
Jun. 9, 2006 (EP) 06011960.9

(51) Int. Cl. G01N 33/53; C12M 1/34; C40B 60/12

(52) U.S. Cl. 435/7.72; 435/287.9; 506/39

(57) ABSTRACT

The present invention provides a method for the detection of an enzyme E1 in a liquid sample comprising the steps of: a) providing a complex (Sa-Sb-M), wherein (Sa-Sb) is a substrate S of E1 cleavable into Sa and Sb by E1, and M is a marker linked to Sb, b) incubating the sample with the complex under conditions enabling the cleavage of S into Sa and Sb by E1, c) separating non-cleaved complex (Sa-Sb-M) from the sample, and d) measuring M in the sample. Furthermore, the present invention further provides kits and devices for the detection of an enzyme E1.

The present invention provides a method for the detection of an enzyme E1 in a liquid sample comprising the steps of providing a complex (Sa-Sb-M), wherein (Sa-Sb) is a substrate S of E1 cleavable into Sa and Sb by E1, and M is a marker linked to Sb, incubating the sample with the complex under conditions enabling the cleavage of S into Sa and Sb by E1, separating non-cleaved complex (Sa-Sb-M) from the sample, and measuring M in the sample. Furthermore, the present invention further provides kits and devices for the detection of an enzyme E1.

The detection of the presence of enzymes in biological samples is often important in diagnostic methods. However, it is often difficult to detect enzymatic activity in a biological sample, because the enzyme is only present in trace amounts or because the natural given enzymatic reaction does not produce an appropriate detectable signal and no corresponding convenient synthetic substrate for detection is available.

This is especially the case in a variety of enzymatic reactions, which are to be assayed in medical clinical tests. In a sample solution unit, such as human plasma, the assayed enzyme is often present mainly in the inactive proenzyme form and only trace amounts of the active enzyme are available for detection. The active form of the enzyme is mostly the clinically more relevant form. Sensitive and specific methods for measuring the active enzyme trace amounts are often very tedious and no convenient direct methods for the differentiation between enzyme and proenzyme forms are available. For example thrombin, activated coagulation factor II (FIIa), is difficult to detect in plasma in the active form. Usually tests are carried out only after converting all available prothrombin to thrombin, as is done in coagulation tests, where the active enzyme coagulates fibrinogen (FI) in plasma (Colman R W, Hirsch J, Marder V J, Salzman E W, eds. Hemostasis and thrombosis: basic principles and clinical practice, 3^{rd} ed. Philadelphia: Lippincott, 1994).

In many cases where the assayed enzyme is hard to detect directly, the reaction is measured indirectly and the presence of an active enzyme is detected through a corresponding biological function. Such is the case for example with active human plasma renin, an aspartic proteinase which has a hypertensive action through its function in the renin-angiotensin system (Sealy J E, Laragh J H: The renin system and its pathophysiology in disease. Seldin D W, Giebisch G, eds. The regulation of sodium and chloride balance. New York: Raven Press, 1989: 193-231). For quantitative determinations renin is injected intravenously into test animals and its pressor effect on blood pressure is measured (Smeby R R and Bumpus F M, Methods in Enzymology Vol. 19, 1970, 699-706).

The aspartic proteinases belong to a category of enzymes involved in a number of major diseases such as the HIV-proteinases in AIDS, the cathepsins in tumorigenesis and the stomach enzyme pepsin, which is responsible for tissue damage in peptic ulcer disease (Cooper J B, Aspartic proteinases in disease: A Structural Perspective. Current Drug Targets, 2002, 3.155-173). For many aspartic proteinases, a convenient method for the detection of their enzymatic activity is not known since their proteolytic and peptidolytic reactions produce no significant changes in the monitored signal. A variety of assay systems have been developed to detect and determine the concentration of inactive proenzymes, active enzymes and the products of their reactions in a test sample.

The immunoassay methods are the most widely used methods to detect these analytes and depend on the binding of an antigen or a hapten, in this case the analyte to a specific labeled antibody (NCCLS. Accessing the quality of immunoassay systems: Radioimmunoassays and enzyme, fluorescence, and luminescence immunoassays; approved guideline. NCCLS Document I/LA23A, Vol. 24 No 16. Villanova: NCCLS 2004).

In conventional immunoassay methods such as FIA, fluoroimmunoassay (Hemmilä I, Fluoroimmunoassays and immunofluorometric assays. Clin Chem 1985; 31: 359-70) fluorochromes are used as labels. In EIA, enzyme immunoassay (Jenkins S H. Homogeneous enzyme immunoassay. J Immunol Meth 1992; 150:91-7) antibodies against the analyte are conjugated with a label enzyme. In RIA, radioimmunoassay (NCCLS. Assessing the quality of radioimmunoassay systems NCCLS Document Order Code LA 1-A Vol. 56 Villanova: NCCLS, 1985) radioisotopes are used as labels. The RIA requires special precautions, because radioactive substances are used and is therefore not as widespread in its use as for example the FIA and EIA. This is true for all methods of detection involving radioactive substances, in comparison to equal methods of detection involving no radioactivity. Being able to offer a method of detection, which contains no radioactive marker, represents therefore a clear advantage.

The sandwich immunoassay method ELISA, enzyme-linked immunoassay (Butler J E., Methods in Enzymol. 1981; 73:482-523, Crowther J R, Methods Mol. Biol. 1995; 42:1-128) is based on trapping the analyte as an antigen by an antibody precoated on a solid phase. A detectable signal is produced by adding a second antibody which binds to the immobilized antigen-antibody complex and which is labeled with an enzyme able to give a detectable signal.

All these immunoassay methods have one basic aspect in common, which is that a substance, the analyte as such, is targeted and antibodies are raised to detect it, thereby measuring its concentration. Often, in fact the proenzyme is be targeted and determined as the antigen. In some other cases, the active enzyme as such is targeted, whereby the active site of the tested enzyme is taken as an antigenic target for raising the specific appropriate antibodies. This is a very tedious and complex process due to the strong similarity between inactive and active enzymes. Furthermore, the product of an enzymatic reaction can be used as an antigen. For example, the activity of renin in human plasma, is determined with an immunoassay test, whereby Angiotensin I, the product of the reaction of renin and plasma Angioten-sinogen, is determined (Ikeda I, Iinuma K, Takai M et al, J Clin Endocrinol Metab 1981; 54:423).

These immunoassay test methods have usually other limitations such as the interference of non-specific antigen reactions with other compounds present in a test solution such as for example human plasma, resulting in a loss of assay sensitivity. Therefore there is a need for improving these immunological techniques, when applied for the detection of enzymes and their activities.

Another major method for measuring enzymatic reactions is the use of small natural or synthetic substrates, which carry an integrated label that is transformed during reaction, thereby producing a signal. The markers mostly used are chromophores, fluoromeres or radioactive isotopes. Such labeled substrates produce often too small signals for the detection of trace amounts of enzymes. Furthermore, the non-processed small natural or synthetic substrate remains in the reaction solution and its signal often interferes with the processed small natural or synthetic product, thereby decreasing the net change in signal intensity. Therefore, here too there is a need for improving the available techniques to produce quick, sensitive and convenient methods for the detection of enzymatic reactions, especially for the detection of trace amounts of enzyme reactivity. In a first aspect, the invention provides a method for the detection of an enzyme E1 in a liquid sample comprising the steps of
a) providing a complex (Sa-Sb-M), wherein (Sa-Sb) is a substrate S of E1 cleavable into Sa and Sb by E1, and M is a marker linked to Sb,
b) incubating the sample with the complex under conditions enabling the cleavage of S into Sa and Sb by E1, thereby generating complex Sb-M,
c) separating non-cleaved complex (Sa-Sb-M) from the sample, and
d) measuring M in the sample.

Preferably, the separating of step c) does not involve a magnetic field. By the method of the invention, it is possible to detect an enzyme activity in the liquid sample with great sensitivity. This is due to the separation of processed and non-processed substrate after the cleaving reaction, allowing thus the measurement of that marker in the sample bound to the cleaved substrate.

The invention may be exemplified by the complex comprising components “A” and “B”. Accordingly, the complex comprising two components “A” and “B” is denoted as (A-B). In this complex, A may be liked to B in a covalent or non-covalent manner. Furthermore, A may be linked to B either directly or via other components, such as a linker molecule.

Preferably, complex Sb-M is released into the liquid phase as a result of the cleavage of step b). This means that before having reacted with E1 the complex has not been dissolved or suspended in the liquid phase. For example, the complex may have been bound to a solid support or carrier such as a reaction vessel. The complex may be attached as detailed below in connection with the reaction device.

In one preferred embodiment of the invention the complex Sa-Sb-M is immobilized during steps a) to c) and optionally d). “Immobilized” in this context means that the complex is attached to an inert, insoluble material such as a support or surface. For example, the complex Sa-Sb-M provided in step a) may be bound to a surface of a reaction chamber in which the reaction takes place. The complex is covalently bound to the surface. The support or surface may also be e.g. part of a reaction device as defined below. Preferably, essentially all complexes are attached to the same support at a defined position, which allows for convenient separation of non-cleaved complex (Sa-Sb-M) from the sample.

It is also comtemplated that the steps b) and d) are performed at distinct sections or distinct positions in the same reaction chamber. A reaction device as defined below may be used in order to carry pout this method and the method may be defined as described in connection with the reaction device. For example, both S may be attached at a defined position in the reaction vessel and a substance need for the detection of M may be positioned at a different and distinct position. The sample is first reacted at the position of S, wherein Sb-M is released into the sample. Then the sample is transferred to position, at which the substance need for the detection of M is located. Reaction of M with this substance generates a detectable signal, therefore, being indication of the presence of E1.

According to the invention, the sample may be from any natural or artificial sources containing the enzyme to be detected. Preferably, the sample may be derived from human blood, human plasma, human serum, human urine, human secrete fluids, animal blood, animal plasma, animal serum, animal urine, animal secrete fluids, fluid human tissue extracts, fluid animal tissue extracts and other fluid tissue extracts, bacterial extract solutions, plant fluids, fluid plant tissue extracts, viral extract solutions or from fluids from artificially or genetically modified or otherwise engineered sources.

The enzyme to be detected in the method of the invention may be any enzyme capable of cleaving a substrate. This includes that the enzyme E1 may be a hydrolytic enzyme or a phosphorolytic enzyme.

In a further preferred embodiment, the hydrolytic enzyme is a peptide hydrolase, lipase, glycosylase, nuclease or other hydrolase.

Regarding the peptide hydrolases, E1 may be selected from the group consisting of aminopeptidases, dipeptidases, dipeptidyl-peptidases, tripeptidyl-peptidases, peptidyl-dipeptidases, serine-type carboxypeptidases, metallocarboxypeptidases, cystein-type carboxypeptidases, omega peptidases, serine endopeptidases, cysteine endopeptidases, aspartic endopeptidases, metalloendopeptidases, threonine endopeptidases, threonine proteases, endopeptidases of unknown mechanism, glutamic acid proteases and other peptide hydrolases including: chymotrypsins, subtilisins, extra cellular matrix proteases alpha/beta hydrolases, signal peptidases, proteasome hydrolases, cathepsins, caspases, secretases, calpains, proteasomes plasmepsins, collagenases, carboxypeptidases, plasma coagulation factors, complement system components, elastases, gelatinases, matrylysins, trypsins, kallikreins, renins, pepsins and other peptide hydrolases.

With respect to glycosylases, E1 may be a glycosidase hydrolyzing, O-, S- or N-glycosylyl compounds. For example, E1 may be a P-glycosylase, a maltase, a cyclodextrine glycosyltransferase, an α-1,6-glycosydase, a cellulose or a lactase.

Regarding the nucleases, E1 may be selected from the group consisting of DNases, ribonucleases, restriction endonucleases type I, II and III, nucleotidases, exonucleases, exoribonucleases, exodeoxyribonucleases and other enzymes hydrolyzing mononucleotides, DNA, RNA, polynucleotides and other synthetic substrates.

Furthermore, E1 may be another hydrolase, e.g. selected from the group consisting of Carboxylic ester hydrolases, thiolester hydrolases, phosphoricmonoester hydrolases, phosphoric diester hydrolases, triphosphoric monoester hydrolases, sulfuric ester hydrolases, diphosphoric monoester hydrolases, phosphoric trister hydrolases, thioether hydrolases, trialkylsulfonium hydrolases, ether hydrolases, linear amide hydrolases, cyclic amide hydrolases, linear amidine hydrolases, cyclic amidine hydrolases, nitriles hydrolases, phosphor-anhydride hydrolases, sulfonyl-anhydride hydrolases, acid anhydride hydrolases, GTP-hydrolases, keton hydrolases, c-halide hydrolases, phosphor-nitrogen hydrolases, sulfur-nitrogen hydrolases, carbon-phosphor hydrolases, sulfur-sulfur hydrolases and carbon-sulfur hydrolases.

According to the invention, “S” is a substrate of the enzyme E1 to be detected in the method of the invention. This substrate comprises two parts, namely Sa and Sb, which are covalently linked to each other. Cleavage of S by E1 results in Sa and Sb, both potentially linked to other binding partners (as M for Sb and A for Sa as explained below).

The skilled person will appreciate that the nature of the substrate S will depend on the nature of the enzyme E1 to be detected in the method of the invention.

In the case that E1 is a peptide hydrolase, the following substrates may be used for detecting the following enzymes:

H-Gly-Lys-OH, H-Pro-Arg-OH, H-Val-Arg-OH, H-Val-Pro-Arg-OH, H-Phe-Val-Arg-OH, H-Phe-Arg-OH, H-Phe-Pro-Arg-OH, H-Gly-Pro-Lys-OH, H-Gly-Gly-Arg-OH, H-Gly-Pro-Arg-OH and any derivatives of these for Coagulation Factor Ia (Thrombin),
H-Asp-Arg-Val-Tyr-Ile-His-Pro-Phe-His-Leu-Leu-Val-Tyr-Ser-OH, H-Asp-Arg-Val-Tyr-Ile-His-Pro-Phe-His-Leu-Val-Ile-His-Asn-OH, H-Asp-Arg-Val-Tyr-Ile-His-Pro-Phe-His-Leu-Leu-Tyr-Tyr-Ser-OH, H-Asp-Arg-Val-Tyr-Ile-His-Pro-Phe-His-Leu-Leu-Val-Tyr-Ser-OH, H-Asp-Arg-Val-Tyr-Ile-His-Pro-Phe-His-Leu-Val-Ile-His-OH, H-Asp-Arg-Val-Tyr-Ile-His-Pro-Phe-His-Leu-Val-Ile-His-Asn-OH, H-Asp-Arg-Val-Tyr-Ile-His-Pro-Phe-His-Leu-Val-Ile-His-Ser-OH, H-Ile-His-Pro-Phe-His-Leu-Val-Ile-His-Thr-OH, H-Arg-Pro-Phe-His-Leu-Leu-Val-Val-Tyr-OH, H-Pro-Phe-His-Leu-Leu-Val-Tyr-Ser-OH
and any derivatives of these for Renin,
H-Glu-Gly-Arg-OH and any derivatives of it for Coagulation Factor Ixa,
H-Ile-Glu-Gly-Arg-OH, H-Leu-Gly-Arg-OH, H-Gly-Pro-Lys-OH and any derivatives of these for Coagulation Factor Xa,
H-Glu-Ala-Arg-OH, H-Phe-Ser-Arg-OH, H-Pyr-Pro-Arg-OH and any derivatives of these for Coagulation Factor XIa,
H-Phe-Arg-OH, H-Gln-Gly-Arg-OH, H-Glu-Gly-Arg-OH, H-Ile-Glu-Gly-Arg-OH and any derivatives of these for Coagulation Factor XIIa,
H-Met-Leu-Ala-Arg-Arg-Lys-Pro-Val-Leu-Pro-Ala-Leu-Thr-Ile-Asn-Pro-OH and any derivatives of it for Anthrax Lethal Factor,
H-Asp-Glu-Val-Asp-OH, H-Asp-Met-Gln-Asp-OH, H-Asp-Glu-Val-Asp-Ala-Pro-Lys-OH, H-Asp-Gln-Met-Asp-OH and any derivatives of these for Casapase-3,
H-Glu-Asp-Lys-Pro-Ile-Leu-Phe-Phe-Arg-Leu-Gly-Lys-Glu-OH, H-Asp-Arg-Val-Tyr-Ile-His-Pro-Phe-His-Leu-Leu-Val-Tyr-Ser-OH, H-Arg-Gly-Phe-Phe-Leu-OH, H-Arg-Gly-Phe-Phe-Pro-OH, H-Gly-Lys-Pro-Ile-Leu-Phe-Phe-Arg-Leu-Lys-Arg-OH, H-Phe-Ala-Ala-Phe-Phe-Val-Leu-OH, H-Phe-Gly-His-Phe-Phe-Ala-Phe-OH, H-Phe-Ser-Phe-Phe-Ala-Ala-OH, H-Pro-Thr-Glu-Phe-Phe-Arg-Leu-OH and any derivatives of these for Cathepsin D,
H-Nal-Abu-Phe-Abu-Abu-Nal-OH and any derivatives of it for Feline Immunodeficiency Virus (FIV) protease,
H-Asp-Glu-Asp-Glu-Glu-Abu-Ser-Lys-OH, H-Glu-Ala-Gly-Asp-Asp-Ile-Val-Pro-Cys-Ser-Met-Ser-Tyr-Thr-Trp-Thr-Gly-Ala-OH and any derivatives of these for Hepatitis C Virus (HCV) NS3 protease,
H-Arg-Gly-Val-Val-Asn-Ala-Ser-Ser-Arg-Leu-Ala-Lys-OH, H-Arg-Gly-Val-Val-Asn-Ala-Ser-Ser-Arg-Leu-Ala-OH and any derivatives of these for Human Cytomegalovirus (CMV) protease (Assemblin),
H-Ala-Pro-Gln-Val-Leu-Phe-Val-Met-His-Pro-Leu-OH and any derivatives of it for Human T-Cell Leukemia Virus Type I (HTLV-I) protease,
H-Phe-Arg-OH, H-Ile-Glu-Gly-Arg-OH, H-Pro-Phe-Arg-OH, H-Val-Leu-Arg-OH and any derivatives of these for Kallikrein,
H-Val-Ser-Val-Asn-Ser-Thr-Leu-Gln-Ser-Gly-Leu-Arg-Lys-Met-Ala-OH and any derivatives of it for SARS protease,
H-Ala-Ala-Pro-Phe-OH, H-Ala-Ala-Phe-OH, H-Gly-Gly-Phe-OH, H-Ala-Ala-Pro-Met-OH, H-Ala-Ile-Pro-Met-OH, H-Ser-Glu-Val-Asn-Leu-Asp-Ala-Glu-Phe-OH, H-Phe-Leu-Phe-OH, H-Val-Pro-Phe-OH and any derivatives of these for Chymotrypsin, H-Gln-Ala-Arg-OH, H-Gln-Gly-Arg-OH, H-Val-Gly-Arg-OH, H-Ala-Ala-Pro-Arg-OH, H-Gly-Gly-Arg-OH, H-Ala-Ala-Pro-Lys-OH, H-Glu-Gly-Arg-OH and any derivatives of these for Trypsin, or
H-Gly-Gly-Phe-Phe-OH, H-Leu-Ser-Phe-Nle-Ala-Leu-OH, H-Phe-Ala-Ala-Phe-Phe-Val-Leu-OH, H-Phe-Gly-His-Phe-Phe-Ala-Phe-OH, H-Pro-Thr-Glu-Phe-Phe-Arg-Leu-OH, H-His-Phe-Phe-OH, H-His-Phe-Trp-OH, H-His-Phe-Tyr-OH, H-His-Tyr-Tyr-OH and any derivatives of these for Pepsin.

In case that E1 is a glycosylase, e.g. dextrin, maltodextrin, cellulose or any other polysaccharide or synthetic substrate of glycosylasis may be used. For example, the following substrates may be used in order to detect the following enzymes:

Dextrin or any derivative of it for cyclodextrin glycosyltransferases,
glycogen or any derivative of it for α-1,6-glucosidases, cellulose or any derivative of it for cellulases and lactose or any derivative of it for lactases.

If E1 is a nuclease, examples for substrates and enzymes include:

5′-C-C-G-C-T-C-3′
3′-G-G-C-G-A-G-5′

and any of its derivatives for AccBSI restriction endonucleases, with either the 5′-3′ or 3′-5′ strand incorporated in the substrate embodiment and the complementary polynucleotide strand associated to it through hydrogen bonds,

5′-G-T-A-T-A-C-3′
3′-C-A-T-A-T-G-5′

and any of its derivatives for Bst1107I restriction endonucleases,
with either the 5′-3′ or 3′-5′ strand incorporated in the substrate embodiment and the complementary polynucleotide strand associated to it through hydrogen bonds,

5′-A-G-C-T-3′
3′-T-C-G-A-5′

and any of its derivatives for AluI restriction endonucleases,
with either the 5′-3′ or 3′-5′ strand incorporated in the substrate embodiment and the complementary polynucleotide strand associated to it through hydrogen bonds,

5′-A-A-G-C-T-T-3′
3′-T-T-C-G-A-A-5′

and any of its derivatives for HindIII restriction endonucleases,
with either the 5′-3′ or 3′-5′ strand incorporated in the substrate embodiment and the complementary polynucleotide strand associated to it through hydrogen bonds,

5′-G-A-A-T-T-C-3′
3′-C-T-T-A-A-G-5′

and any of its derivatives for EcoRI restriction endonucleases,
with either the 5′-3′ or 3′-5′ strand incorporated in the substrate embodiment and the complementary polynucleotide strand associated to it through hydrogen bonds,

5′-C-C-C-G-G-G-3′
3′-G-G-G-C-C-C-5′

and any of its derivatives for SmaI restriction endonucleases,
with either the 5′-3′ or 3′-5′ strand incorporated in the substrate embodiment and the complementary polynucleotide strand associated to it through hydrogen bonds.

If E1 is another hydrolase, the substrate may contain one of the following structures:

Carboxylic ester bonds/structures, thiolester bonds/structures, phosphoricmonoester bonds/structures, phosphoric diester bonds/structures, triphosphoric monoester bonds/structures, sulfuric ester bonds/structures, diphosphoric monoester bonds/structures, phosphoric triester bonds/structures, thioether bonds/structures, trialkylsulfonium bonds/structures, ether bonds/structures, linear amide bonds/structures, cyclic amide bonds/structures, linear amidine bonds/structures, cyclic amidine bonds/structures, nitrile bonds/structures, phosphor-anhydride bonds/structures, sulfonyl-anhydride bonds/structures, acid-anhydride bonds/structures, GTP, keton bonds/structures, c-halide bonds/structures, phosphor-nitrogem bonds/structures, sulfur-nitrogen bonds/structures, carbon-phosphor bonds/structures, sulfur-sulfur bonds/structures, carbon-sulfur bonds/structures, such as:
Phospholipids, glycerophospholipids, sphingolipids, lipoproteins, ceramides, sphingomyelins, glycolipids, glycosphingolipids, cerebrosides, galactocerebrosides, glucocerebrosides, gangliosides, diglycerids, triglycerides, terpenoids, steroids, or any other lipids or synthetic substrates containing these bonds/structures.

Specific examples include:

Phosphatidylcholine or any derivative of it for phospholipase D,
GM2 ganglioside or any derivative of it for β-N-acetylhexosaminidase,
Phosphatidylinositol or any derivative of it for phospholipase C,
Triacylglycerol or any derivative of it for triacylglycerol lipases.

This list of enzymes and corresponding substrates available for the method of invention is exemplary and not exhaustive.

In a preferred embodiment of the invention, Sb is covalently bound to M via a binding moiety L2. This binding moiety may be any chemical entity enabling the binding of Sb to M. In its simplest form, L2 may be a chemical bond. Preferably, L2 contains at least one atom.

In a preferred embodiment, the binding moiety L2 is a linker molecule. The nature of this linker molecule is discussed below.

Methods for linking Sb covalently to M, thereby forming the complex (Sa-Sb-M), are known in the art. The same applies to all other complexes described in the context of the present invention. With respect to that linking, in a preferred embodiment the following general considerations may apply:

In a first step, usually one of the partners is activated. Such activation may be performed using glutaraldeyde, cyanogens bromide, hydrazine, bisepoxiranes, benzoquinone, periodate and other substances, depending on the chemical nature of the partner.

Next, a linker may be conjugated to said activated partner, again by methods known in the art. In this context, it is preferred that the linker is also activated at two sides.

In a second step the activated partner or the activated attached linker is conjugated to the other binding partner.

In this context, the activation and binding of one partner to another may proceed also in one step.

According to the invention, Sa may further be linked to an anchor entity A, resulting in a complex (A-Sa-Sb-M), such that after the cleavage in step b) at least the complexes (Sa-A) and (Sb-M) are formed.

Consequently, in this preferred embodiment of the invention, the substrate S is further linked to an anchor entity A. This anchor entity A is linked to Sa and not to Sb. After the cleavage, A remains linked to Sa, while M remains linked to Sb. Consequently, in this preferred embodiment of the invention, after cleavage with E1, at least two complexes and, potentially, three complexes remain in the sample, namely the non-cleaved complex (A-S-M), the complex (Sa-A), and the complex (Sb-M). If A is used to separate non-cleaved complex from the sample, this means that by removing the complexes comprising A, the complex (Sb-M) is enriched, which allows the detection of the cleaved substrate S. In this context, the skilled person will appreciate that the more (Sb-M) is enriched, the clearer the signal (e.g. also over a control reaction) will be.

In a preferred embodiment, Sa is covalently bound to A via a binding moiety L1 and/or Sb is covalently bound to M via a binding moiety L2. Binding moiety L1 and/or binding moiety L2 may be any chemical entity enabling the binding of Sa to A and Sb to M, respectively. In its simplest form, L1 and/or L2 may be a chemical bond. Preferably, L1 and/or L2 contain(s) at least one atom. More preferably both L1 and L2 are binding moieties as defined above.

In a preferred embodiment, binding moiety L1 or binding moiety L2 is a linker molecule. More preferably, L1 and L2 are linker molecules.

In the context of the invention, principally all suitable linker molecules can be used as L1 or L2. For example, the linker molecule may be an alkane, alkene, alkyne, an acryl, a lipid, polysaccharide, polynucleotide, peptide molecule or a synthetic polymer.

The linker molecule may be substituted in order to enable to binding to Sa, Sb, A or M, respectively. Such methods are known in the art.

In a preferred embodiment, the linker molecules are long enough to guarantee that interaction with one part of the complex, e.g. with A or M, leaves the other parts of the complex unaffected. Furthermore, it is preferred that the linker molecules are long enough to ensure that different parts of the complex, e.g. A or M, do not interfere with the cleaving process of the enzymatic reaction. Preferably, the linker molecules have a linear structure, with preferably a minimum length of two atoms, more preferably between 20 and 30 atoms, the length may depend on the nature of the substrate and the structure of the active site of the enzyme E1.

Consequently, in an especially preferred embodiment of the method of the invention, a complex (A-L1-Sa-Sb-L2-M) is used for detecting E1 in a liquid sample, wherein both L1 and L2 are linker molecules as defined above.

As discussed above, in step b) of the method of the invention, the sample is incubated with the complex under conditions enabling the cleavage of S by E1. The products of such cleavage are Sa (in a preferred embodiment the complex A-Sa) and a complex of Sb and M (Sb-M). Conditions enabling the cleavage of S by E1 will depend on the individual enzyme E1 to be detected and are principally known in the art (Methods in Enzymology: Proteolytic Enzymes Vol. 19: p. 3-1042, 1970, Edited by Laszlo Lorand and Part B, Vol. 45; p. 3-939, 1976, Edited by Gertrude E. Perlmann and Laszlo Lorand).

In the next step of the method of the invention, non-cleaved complex is separated from the sample. This can be performed by several methods, including the use of binding molecules, e.g. antibodies, which specifically bind S but not Sb. In a preferred embodiment of the invention, the anchor entity A is used to separate non-cleaved substrate S from the sample.

In the following, several preferred embodiments will be discussed in order to demonstrate how an anchor entity A can be used for that purpose. Removal of (A-S-M) may result also in a removal of (A-Sa), further increasing the purity of (Sb-M).

In one possibility, A is the high molecular soluble compound, preferably with a molecular weight of 100 kDa or higher. In this context, A may be a dextran, protein, gelatine, polyglycan, polyxylan, amylase, amylopectin, galactan or polynucleic acid. The person skilled in the art will be aware of any further bulky molecules which can also be used in that context.

In a further preferred embodiment in this context, A is or further comprises a dye. This has the advantage of enabling a quick control of leakage and the location of the anchor molecule A during separation of A from M. Preferably, A is Dextran Blue with a molecular weight of 100 kDa or higher.

Preferably, in this context, the separation of non-cleaved complex from the sample is performed by using molecular weight cut-off filtration, e.g. by the use of a molecular sieve. The anchor entity A is retained, while the marked part of the complex, with a lower molecular weight than 100 kDa, goes through the cut-off barrier. An unwanted leakage of A through this cut-off barrier may be readily detected through the dye molecule attached to A as described above.

According to the invention, another possibility is that A is part of an insoluble matrix, preferably selected from the group consisting of a Sepharose, cellulose, sephadex, silica gel, acrylic bed or other resin, ceramic bed, Wafer glass, amorphous silicon carabide, castable oxides, polyimides, polymethylmethacrylates, polystyrenes, gold or silicone elastomers and nitrocellulose. Other insoluble matrixes may also be used. In this case, A, and, therefore, non-cleaved complex (A-S-M) can be easily removed from the sample, e.g. by centrifugation or filtration.

In a further preferred embodiment of the invention, non-cleaved S, but not the complex of M and Sb is linked to a removable entity R after step b). In this case R may be an antibody recognizing S, but not Sb.

Preferably, said linking is performed by linking R, preferably in a non-covalent manner, to the anchor entity A linked to Sa as defined and described above.

Consequently, in this embodiment of the invention, non-cleaved complex is removed from the sample by binding A to a removable entity R. In the art, several pairs of compounds are known which can be used for that purpose. For example, A is streptavidin or avidin and R is biotin, A is an antigen and R a specific antibody to said antigen, A is nickel coated surface, and R is a His-tag or A is a magnetic surface and R comprises Fe ions, or vice versa. Further similar non-covalently bound binding pairs are known in the art.

Additionally, R may be linked, preferably covalently bound, to an insoluble matrix either already before the coupling to non-cleaved S (preferably Sa) or during step c), i.e. after the cleaving reaction. This further facilitates the removal of non-cleaved complex (M-S) via the interaction of A and R.

In a preferred embodiment, such matrix is selected from the group consisting of a Sepharose, cellulose, sephadex, silica gel, acrylic bed or other resin, ceramic bed, Wafer glass, amorphous silicon carabide, castable oxides, polyimides, polymethylmethacrylates, polystyrenes, gold or silicone elastomers and nitrocellulose.

In this embodiment of the method of the invention, the non-cleaved complex is separated from the sample by removing the A-R complex.

In a preferred embodiment, the A-R complex is removed by one of the techniques selected from the group consisting of centrifugation, filtration, decantation, adsorption through non-covalent forces, use of magnetic force, and steady rinsing.

After non-cleaved (S-M) complex has been removed from the sample, M is measured in the sample according to step d) of the method of the invention. The concrete nature of such measurement will depend on the nature of the marker M.

In a preferred embodiment, M is an enzyme E2 or a chemical compound. In a more preferred embodiment, M is an enzyme E2. Still more preferably, the enzyme is capable of generating a detectable signal under suitable conditions. The signal may be any chemical or physical change such as a change in temperature, pH value, concentration of a molecule or ion, color change, increase or decrease fluorescence, altered conductivity etc.

Preferably, an enzyme E2 is used which does not interfere with the reaction of E1 with S. Preferably, E2 belongs to another class than E1, which minimizes the risk that the activities of both enzymes do interfere.

This enzyme E2 may be a peroxidase, a phosphatase, a luciferase, a monooxygenase, beta-galactosidase, or acetyl cholinesterase.

In a preferred embodiment, E2 is selected from the group consisting of horse radish peroxidase (HRP), alkaline phosphatase (AKP), acidic phosphatase, photinus-luciferin 4-monooxygenase, renilla-luciferin 2-monooxygenase, cypridinia-luciferin 2-monooxy-genase, watasenia-luciferin 2-monooxygenase, oplophorus-luciferin 2-monooxygenase, beta-galactosidase, and acetyl cholinesterase.

In a preferred embodiment, E2 is measured by incubating the sample with a substrate S2 for E2 and measuring the reaction of E2 with S2. This is known to the person skilled in the art.

In a further preferred embodiment, the chemical compound is a molecular tag with a molecular weight of at least 100 Da. The concentration of the cleaved molecular tag in the reaction solution may correspond to the reactivity of the corresponding substrate The molecular tag may be measured by molecular sieve chromatography or mass spectrometry according to methods known by the person skilled in the art (Methods in Enzymology Vol. 402, p. 1478, 2005: Biological Mass Spectrometry, Edited by A. L. Burlingame).

In a further preferred embodiment, M is a dye substance, chromophore, or fluoromere. Then, M may be measured by detecting the dye substance, chromophore, or the fluoromere according to methods known in the art, e.g. by spectroscopy.

In a further preferred embodiment, the chemical compound is an organic molecule with a functional group such as an alcohol, aldehyde, amine, dibromoamine, thiol, a pH dye indicator such as phenolphthalein (3,3-Bis(4-hydroxyphenyl)-1(3H)-isobenzofuranone), or glucose, or any other functional group. These chemical functional groups can be processed further to produce a strong signal, without intervening with the tested enzyme reaction. The detection of the functional group thiol for example can be carried out by modification with (DTNB) 5,5′-Dithio-bis-(2-nitrobenzoic Acid), known as Ellman's Reagent, resulting thus in a strong yellow chromophore, which is measured by its absorbance at 412 nm.

In a preferred embodiment, the chemical compound is transformed further to produce a signal. An example for this is Phenolphthalein, which when transformed by a pH change up to 10 produces an intense color signal at 374-552 nm, or the chemical compound dibromoamine, which when transformed by reaction with indigo carmine produces a signal at 608 nm. Furthermore, when M is a glucose residue it can be determined using glucose oxidase techniques. In this case glucose is oxidized, enzymatically to gluconic acid and hydrogen peroxide by glucose oxidase. Hydrogen peroxide is then, e.g., determined enzymatically with horseradish peroxidase.

In a preferred embodiment, two or more complexes (Sa-Sb-M) with different S for different E1 and different M are provided, thereby enabling the detection of these E1. Furthermore, two or more complexes (Sa-Sb-M) with different S for E1 and different M are provided, thereby enabling the testing of the reaction of E1 with multiple substrates in a sample. For these embodiments, it is important that the individual components do not interfere with each other.

Depending on how M is detected, it may be suitable to perform controls, e.g. by not adding the substrate complex or by not removing non-cleaved S. Such control methods are known to the person skilled in the art. If a control is performed, in step d) of the method of the invention the result obtained may be also compared to the result of said control.

The invention further refers to a kit comprising the complex (A-L1-Sa-Sb-L2-M), with A, L1, Sa, Sb, L2 and M as defined above.

As explained above, such a kit is especially useful for detecting an enzyme E1, the substrate thereof is (Sa-Sb), in a liquid sample. All embodiments defined above with respect to the method of the invention also apply to the kit of the invention.

In a preferred embodiment, the kit of the invention further comprises a removable entity R as defined above, and, even more preferred, buffer solutions.

A kit of the invention is exemplified in Example 1. As further examples, additional kits are given in Example 2.

The invention is preferably implemented by the reaction device of one of claims 46-57 or by the array one of claims 58-59. The reaction device is adapted to carry out the following method of detecting an enzyme in a liquid sample:

A complex (Sa-Sb-M) is provided in the reaction device, wherein (Sa-Sb) is a substrate S of E1 cleavable into Sa and Sb by E1, and M is a marker linked to Sb, wherein M comprises enzyme E2. The sample is incubated in the reaction device with the complex under conditions enabling the cleavage of S into Sa and Sb by E1. In a second incubation step E2 is reacted with S2 to produce a detectable signal. The concept underlying the implementation is to separate the enzyme E2 of uncleaved substrate S1 (comprising Sa-Sb-M) from substrate S2 by attaching substantially the complete amount of substrate S1 to a surface. In this way, the location of substrate S1 can be defined by defining the location of the surface. The surface carrying S1 is referred to as the first surface. In order to avoid any contact between substrate S2 and uncleaved Sa-Sb-M, basically two general mechanisms can by used for implementing the separation assembly defined in the claims.

The first mechanism is to ensure that substrate S1 is removed from the sample solution, if S2 is soluble in the sample fluid. This can be implemented by a stopper, a locking mechanism or similar means for blocking the sample fluid, if S1, i.e. the surface carrying S1, is present in the fluid. As an example, the first surface of S1 can be connected to a handle or a grip chamber for handling the surface of S1. This grip extends into the reaction thereby providing a spacer or a stopper, which prohibits the insertion or application of S2 (provided as solution or on a carrier) into the sample fluid or into the processing chamber. In an alternative example, a mechanical connection, e.g. a lever, connects the first surface carrying S1 and a second surface on which substrate S2 is located, e.g. a carrier. The lever actively moves the first surface out of the sample fluid in an active way, if the second surface carrying S2 is moved into the sample fluid. Of course, other mechanisms connecting the first surface to the second surface can be used which move the second surface in a direction opposed to the direction the first surface is moved. In one embodiment of the invention, the first surface is moved by a first actuator and the second surface is moved by a second actuator, both actuators being controlled by a control, the control implementing the mutually opposed movements, which can be performed simultaneously or sequentially (sequence: step (1): removal of the first surface, step (2), performed after step (1): introducing the second surface into the reaction chamber). The control can be implemented as software on a personal computer.

The second mechanism is to ensure that substrate S1 and substrate S2 are not provided at the same location, if S2 is substantially insoluble. This can implemented by a second surface on which substrate S1 is bound and by a spacer mechanism similar to the implementations of the first mechanism described above. In general, the spacer mechanism of the second mechanism provides a fixed distance, e.g. by a positive or non-positive connection. Alternatively, the spacer mechanism provides a variable distance with a lower limit, the lower limit ensuring the separation of the first and the second surface. The lower limit of the variable distance can be defined as a contacting threshold. If the distance is greater than the threshold, the first surface and substrate S2 are isolated or separated from each other. Thus, the first surface does not contact substrate S2, if the distance exceeds the lower limit, i.e. the contacting threshold. In an example of a spacer mechanism with a fixed distance, the first and the second surfaces are surfaces of the same carrier, e.g. a test strip or an inner wall of a reaction chamber. Further, the first and the second surface can be surfaces of distinct carriers, the carriers being directly or indirectly bound by a suitable rigid or flexible mechanical connection. In an example of a spacer mechanism with a variable distance with a lower limit, the first surface is located at a lower section or a bottom of a reaction chamber and the second surface is an inner surface of a cap, the cap matching to an opening of the reaction chamber located at an upper section of the reaction chamber. Thus, the minimum distance is defined by the sidewalls of the reaction chamber connecting the lower section and the opening. Of course, the distance between cap and lower section/bottom can be increased by removing the cap from the opening. Further, the first surface can be a surface of a first carrier and the second surface can be a surface of a second carrier. In order to provide a minimum distance, a spacer can be used, the spacer being adapter to contact the first and the second carrier. The spacer can be a spacer removably attached to the carriers or can be a spacer unremovably connected to one or to both carriers or can be a spacer integrally formed with one or both of carriers. In a preferred embodiment, one carrier comprises both surfaces, the surfaces being located on a strip, e.g. adjacent to each other or on opposed sides of the carrier. In this document, the terms “upper” and “lower” are defined by the direction of gravity with regard to a container having a base located at the lower section.

In another embodiment, the first surface and the second surface are surfaces of a reaction chamber, preferably inner surfaces of the reaction chamber. The first surface is located at a section of the reaction chamber distinct from the second surface. Substrate S1 is separated from substrate S2 by the distinct locations of the first and the second surfaces. This way, the sample fluid can be brought into contact with the first surface, i.e. with the first substrate S1. Since S1 is insolubly bound to the first surface, the fluid sample is separated from the uncleaved substrate by separating the sample fluid from the first surface. After the separation of the sample fluid from the first surface, the sample fluid has to be brought into contact with the second surface. Thus, the reaction device comprises a direct fluidic connection between the first surface and the second surface. In this way, the sample liquid can be brought into contact with the first surface, separated from the surface and brought into contact with the second surface by forcing the sample liquid through the fluidic connection.

In one embodiment, the first surface, i.e. the first substrate is located at a lower section or a bottom of the reaction chamber, while the second surface is located at an upper section of the reaction chamber or at a cap, which can be arranged at the upper section of the reaction chamber. The sample fluid is applied into that reaction chamber through the opening located at the upper section, without contacting the second surface. The sample fluid contacts the lower section or the bottom of the reaction chamber, where the first surface is located. Then, the reaction chamber is tilted, for example by an angle of substantially 180° such that the sample fluid is separated from the first surface and is brought into contact with the second surface. Preferably, a cap or another element is used for sealing the opening before tilting the reaction chamber.

Preferably, the reaction chamber is a cylinder formed of the inner surfaces of a cylindrical container, preferably with a continuous cross section, which can be in the shape of a circle, an ellipse or a rectangle. Alternatively, the reaction chamber can be tapered towards the lower section, i.e. towards the bottom of the reaction chamber.

In another embodiment, a plurality of distinct surface sections are comprised by the reaction devise, each of the first surface sections having a distinct, specific enzyme E1′. In this embodiment, the plurality of tests concerning distinct enzymes E1, E1′ or E1n can be carried simultaneously.

In another embodiment, a plurality of reaction devices according to the invention (e.g. 2, 3, 4, 5, 6, 7, 8, 9, 10, 11, 12, 16, 20, 24, 48 or 96) may form an array of reaction devices such as e.g. a multi-well plate. In one embodiment, each reaction device comprises the same substrate, i.e. the same complex Sa-Sb-M specific to the same enzyme E1. In this example, a plurality of liquid samples can be tested in one step. Alternatively, each of the reaction devices of the array has a distinct complex specific to one of the plurality of distinct enzymes E1n. The annex n refers to an index, each index being related to distinct one of all enzymes E1. If distinct enzymes E1n are used, a plurality of liquid samples can be tested in regard to a plurality of distinct, specific enzymes E1. In both embodiments of the array, i.e. an array with a plurality of the identical first substrates S (Sa-Sb-M) or with distinct first substrates Sn (each being specific to a certain enzyme E1n), all substrates S, Sn may comprise the same marker M which includes enzyme E2. Each of the plurality of surfaces is assigned to one substrate S, Sn which works as a multiplier for the same enzyme E1 or distinct enzymes E1n, respectively. The signals caused by the cleavage of S2 are specific to each of the reaction devices of the array. Thus, the cleavage of each of the substrates S2 forms a distinct, specific signal SIGn, wherein the specific signals are demultiplexed or separated from each other by the location of occurrences since each reaction devise is located at a distinct location. The location of a specific signal can be the surface, if substrate S2 is bound to the respective second surface, or can be the volumes of the distinct liquid samples, if the substrate S2 and (and consequently the respective signal) is soluble in the respective liquid sample. In another embodiment of array of reaction devices, the signals SIGn itself are distinguishable from each other. Thus, the signals do not have to be distinguished by the location of occurrence (i.e. by the location of the sample liquid) but can be distinguished by their physical properties, for example wavelength of emitted light, intensity of emitted light, kind of radioactive emission or intension of radioactive emission. In this embodiment, distinct substrates S2 and the respective signals do not have to be separated by separated reaction devices, but can be provided in the same liquid sample. In order to distinguish the plurality of distinct substrates Sn and their respective first enzymes E1n, the markers Mn comprised by their first substrates have to be distinguishable with regard to the distinct substrates S2n. Thus, each reaction device of the array has a dedicated first substrate Sn, a respective first enzyme E1n specific to the first substrate Sn and a respective second substrate S2n generating a specific signal SIGn and being specific to one of the plurality of distinct first enzymes E1n.

According to the invention, the first substrate or substrates S, Sn have to be bound to the respective first surface or surfaces in an unsolublable way. Unsolublable or essentially unsolublable means that the amount of marker M activated (=cleaved) by enzyme E1 can be distinguished from the amount of marker, which is present in the sample liquid due to unwanted transfer of marker M into the sample fluid without the activation by the first enzyme E1. The solubility of the first substrate S should be such that the uncleaved substrate S leads to a signal with less intensity than a first substrate S cleaved by enzyme E1 thereby activating the first substrate S by cleaved marker M.

A similar definition holds for substrate S2, if substrate S2 is bound to the second surface. This means, if substrate S2 is unsoluble, the amount of S2 cleaved by uncleaved substrate S is preferably distinguishable from the amount of substrate S2 cleaved by cleaved substrate S as regards the generated signal. The bond between substrate S and the first surface and the bond between S2 and the second surface, if S2 is unsoluble, can be any suitable bond which is not released in the presence of the liquid sample. The liquid sample can be a solution comprising water and/or alcohol or any other suitable organic or inorganic solvent. Preferably, the liquid sample is aqueous and the bond is a suitable covalent or ionic bond attaching the respective substrate to the corresponding surface.

According to the invention, the first surface and/or the second surface is not distributed on distinct particles, which are movable relative against to each other. Rather, the continuous first surface or the first surfaces or the second surface or the second surfaces are mechanically bound to each other, respectively, such that a force applied to a part or to only one or a subgroup of the respective surfaces directly applies force on the residual surface or surfaces such that the removal of only a part, a section or a subgroup of the surfaces directly leads to the complete removal of the respective surface and, consequently, to the complete removal of the respective substrate. Therefore, the first surface is continuous and covers a total area of at least 0.0025 mm², at least 1 mm² or at least 100 mm². In this way, the first substrate can be moved at once without any means for individually applying a force to the respective substrate and without any means for individually connecting the respective surfaces.

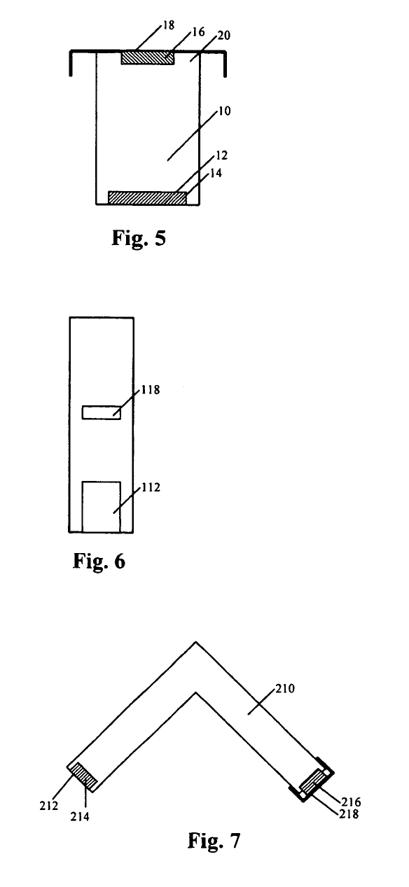

In FIG. 5, a longitudinal cross section of a first embodiment of the reaction device according to the invention is shown. The first embodiment comprises a reaction chamber 10, a first surface 12, on which a first substrate 14 is applied. The embodiment of FIG. 5 further comprises a second substrate 16 applied on a second surface 18. The reaction chamber is formed by a cylindrical container having opening at an upper section 20 and having a bottom, on which the first surface 12 is located. The reaction chamber 10 further implements a direct fluidic connection between the first surface and the second surface. The second surface is part of the inner surface of a cap which is adapted to be applied onto the opening of their reaction chamber. Therefore, if the cap is removed, the opening at the upper section 20 can be used for applying liquid sample into the reaction chamber 10, thereby establishing contact to the first substrate 14 at the bottom 12 of the reaction chamber. Of course, the first surface can be located at another part of the reaction chamber, for example at the lower side walls of the container. If a certain sample solution contains enzyme E1, marker M, together with Sb, is separated from the first substrate and is dissolved in the liquid sample. After cleavage of the first substrate, for example after an incubation time of 15 minutes, the cap can be put onto the opening, thereby sealing the opening and the reaction chamber can be tilted. By tilting the reaction chamber, the sample solution comprising marker M contacts the second substrate 16 on the second surface 18. Further, upon tilting, essentially all of the uncleaved first substrate 14 remains at the first surface. If the second substrate 16 is solublable, a signal will occur in the sample fluid. If the second substrate 16 is unsoluble and bound to the second surface 18, a signal will occur at the second surface. Of course, a signal only occurs, if enzyme E1 is present in the sample solution. If enzyme E1 is not present in the sample solution, the marker M, together with enzyme E2 remains at the first surface 12 and does not lead to a cleavage of the second substrate.

In FIG. 6, the test strip is shown, which is covered by an upper plate, the upper plate having two windows 112, 118. At the first window 112, the first surface and the first substrate are located. At the second window 118, the second surface is located, which is at least partly covered by the second substrate. Between the first surface 112 and the second surface 118 a capillary connection is provided by a stationary phase. Thus, a liquid sample is applied to a first surface 112 for cleaving the first substrate, if enzyme E1 is present in the sample solution. The capillary connection between the first surface 112 and the second surface 118 transports the sample liquid (together with cleaved marker M, if E1 is present) to the second surface 118. Any uncleaved substrate S I remains at the surface 112. At the second surface 118, a signal is produced, if the sample fluid contains the marker M, which cleaves the second substrate located at the second surface. Instead or in combination with a capillary connection, also a connection by diffusion is possible. The diffusion can be amplified and/or directed or forced with an electric field, if the respective marker M or a residue connected therewith is charged.

In FIG. 7, a third embodiment of the reaction device according to the invention is shown, comprising a reaction chamber 210, which forms a direct fluidic contact between the first substrate 214 located at the first surface 212 and the second substrate 216 located at the second surface 218. The direct fluidic connection is curved. In the embodiment shown in FIG. 7, the fluidic connection provides an angle of 90°. Of course, any suitable angle could be used, e.g. 30°, 45°, 60° or 120° or any value between these angles. The second surface 218 is part of a cap, which is used to close the reaction chamber 210. Like in the embodiment of FIG. 5, the fluidic connection between the first and the second surface is a direct fluidic connection. However, the reaction chamber 218 has to be tilted by approximately less than 90°.

The embodiments shown in FIGS. 5, 6 and 7 can have soluble or unsoluble second substrates since the first and the second substrate are separated by the shape of the reaction chamber and by the location of the first and second substrate. Like in FIG. 7, the separation assembly in FIG. 5 is realized by the bond between the first substrate and the bottom of the reaction chamber and by the wall of the container of reaction chamber 10, 210. In FIG. 6, the first and the second substrate are separated by the capillary connection and by the stationary phase provided between the first substrate and the second substrate. In FIG. 6, the distance between the first and the second surface is constant, in contrast to FIGS. 5 and 7, in which the distance between the first and the second surface is defined by the spatial relationship between the cap and the reaction chamber. However, the cap as well as the reaction chamber both ensure the separation between the first and the second substrate.

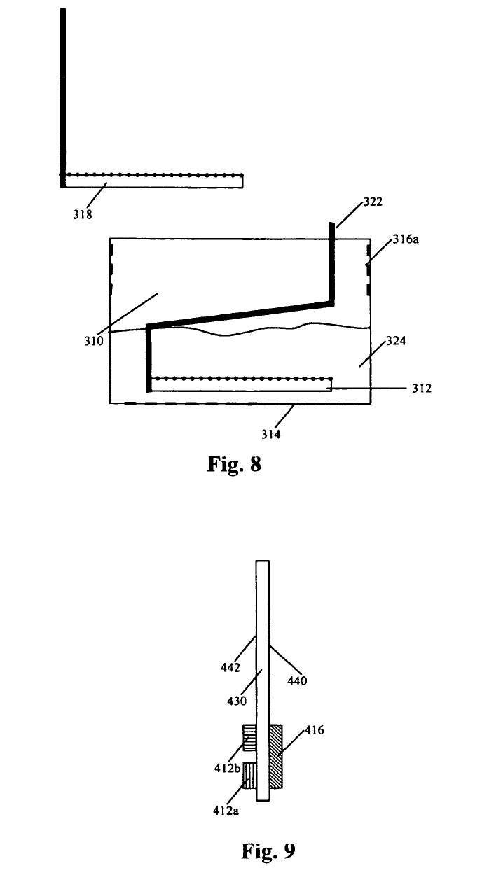

In FIG. 8, the first substrate is located on a first carrier 312, to which a grip or a handle 322 is attached to. The reaction chamber 312 is partially filled with a liquid sample 324, into which the first carrier 312 is completely immersed. The grip 322 forms a spacer element which ensures that a second carrier 318 cannot be brought into contact with the sample solution. The second substrate in the embodiment shown in FIG. 8 is located on the second carrier 318 and can be soluble or unsoluble. Further, the second carrier 318 is attached to another grip for handling the second carrier. Of course, the grip 322 of the first carrier 312 can have any other suitable shape which ensures that a second carrier 318 can not be brought into contact with the sample solution and cannot be introduced into the reaction chamber 310 as long as the first carrier 312 carrying the first substrate is located in the reaction chamber 310.

In a first alternative of the fourth embodiment shown in FIG. 8, the substrate as shown in dotted lines is located on an upper surface on the respective first or second carrier 312, 318. Instead, or in combination therewith, the first substrate can be located at the bottom of the reaction chamber 310 as shown with dashed lines. Further, the second substrate can be located at an upper section of the inner walls of the reaction chamber 310 as shown with reference sign 316a. These alternatives can be combined in any appropriate combination. Thus, the first substrate can be located at the bottom with dashed lines, c.f. reference sign 314, whereby the second substrate is located on the first carrier 318. In this case, the first carrier 312 is not present and the thickness of the second carrier 318 defines the distance between first and second substrate. If the first substrate 314 is located at the bottom of the reaction chamber, the second substrate is preferably not soluble. If the first substrate is located on the first carrier and can be removed from the sample solution, the second substrate located on the second carrier 318 can be soluble or unsoluble.

The embodiment shown in FIG. 8 with a first carrier, on which the first surface and the first substrate is located, is complementary to the embodiment shown in FIGS. 5 to 7 in that the sample solution stays at the same location whereas the first carrier is actively removed from the sample solution.

Also the embodiment shown in FIG. 9 relates to an embodiment, in which the substrate are actively removed from the sample liquid and the sample liquid is not moved. FIG. 9 shows a strip used as a common carrier 430, having a first side 440 and a second side 442. At the second side 442, to distinct first substrates 412a, 412b are located. The first substrates 412a, 412b are specific to distinct enzymes E1, E1′. However, both first substrates comprise the same marker enzyme M with the identical enzyme E2. On the second side 440 of the strip 430, the second substrate 416 is located being specific to enzyme E2 of marker M. Further, the second substrate 416 is not soluble and is bound to the strip 430. The thickness of the strip 430, i.e. the distance between the first and the second side, implements the separation assembly, together with the respective bond between the first substrate and the second substrate to the respective surfaces of the strip 430.

In accordance to the terminology of the claims, the first side 442 comprises two first surfaces, each of which is covered by a specific substrate, and the first side comprises the second surfaces, on which the unsoluble second substrate 416 is located. If the strip is immersed into the liquid sample such that the first substrates and the second substrate contact the liquid sample simultaneously, the cleavage of the second substrate 416 generates a signal, if one or both first substrates 412a, b are cleaved by a respective specific enzyme E1, E1′ in the liquid sample. Thus, the signal provided by the second substrate 416 indicates the presence of at least one of the enzymes E1, E1′. In another embodiment, two second substrates are located on the first side 440, each being specific to one of the enzymes E2, E2′, whereby the first substrates comprise distinct enzymes E2, E2′. In this case, two distinct first substrates are located on the strip.

Further, the field denoted with 412a can be the first substrate, and the field denoted with 421b can be the second substrate of an embodiment without a field 416. In a first step, only the first substrate 412a can be in contact with the sample liquid, and in a subsequent step, the strip can be immersed deeper into the sample liquid providing contact between the second substrate and the sample liquid. These two steps enable incubation time for the first substrate 412a defined by the length of the first step, during which only the first substrate 412a is immersed into the liquid sample. Of course, the same or distinct first and second substrates can be located on the first side of the strip 430. In this case, also the field 416 has to be divided into two fields, the lower field showing the location of another first substrate and the upper field showing the location of another second substrate. As mentioned above, second substrates can be identical for a joint testing procedure. Of course, the gap between the first field 412a and the second field 412b can be adapted to the solubility of the first and/or the second substrates.

The invention is further explained with the help of the figures and examples below, which are not intended to limit the scope of the present invention.

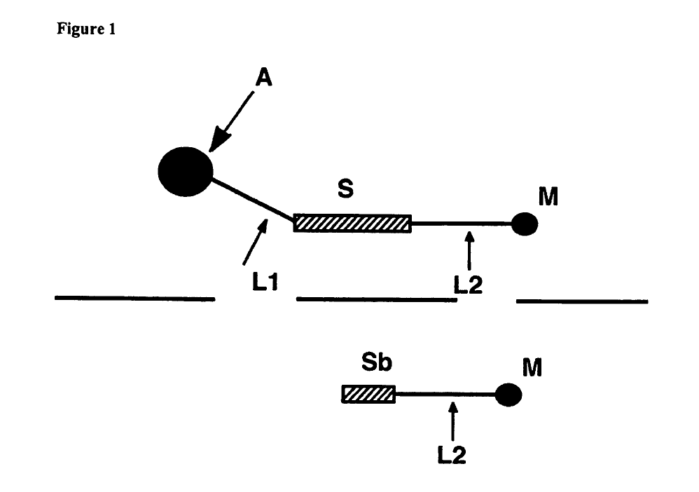

### SHORT LEGENDS TO THE FIGS. 1-9

FIG. 1: Depiction of a possible embodiment of the invention wherein the non-cleaved complex is retracted from the reaction solution via filtration.

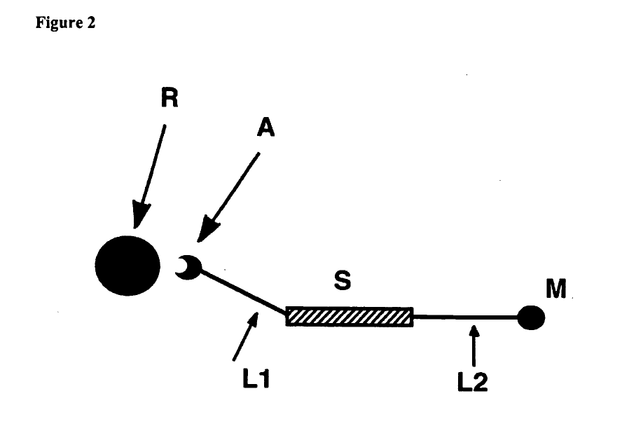

FIG. 2: Depiction of a possible embodiment of the invention wherein the non-cleaved complex is retracted from the reaction solution through the interaction with a retraction molecule R.

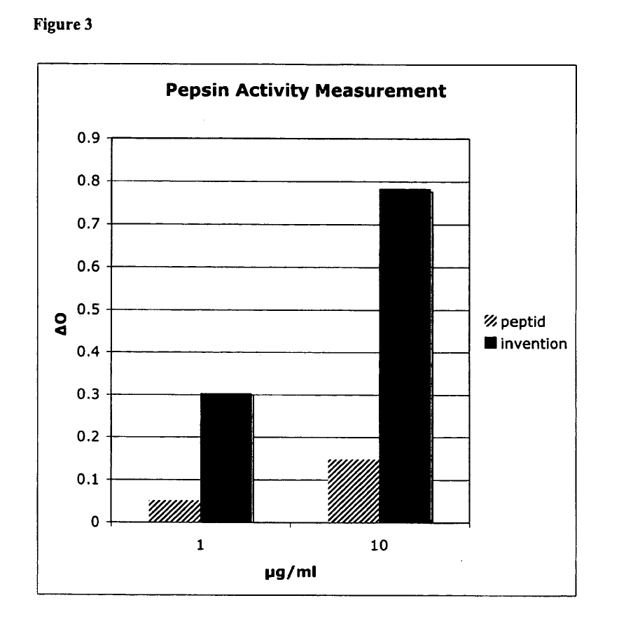

FIG. 3: Difference in Optical Density to the empty control solution after incubation of Pepsin enzyme solutions with the peptide substrate or the embodiment of the invention, as described below.

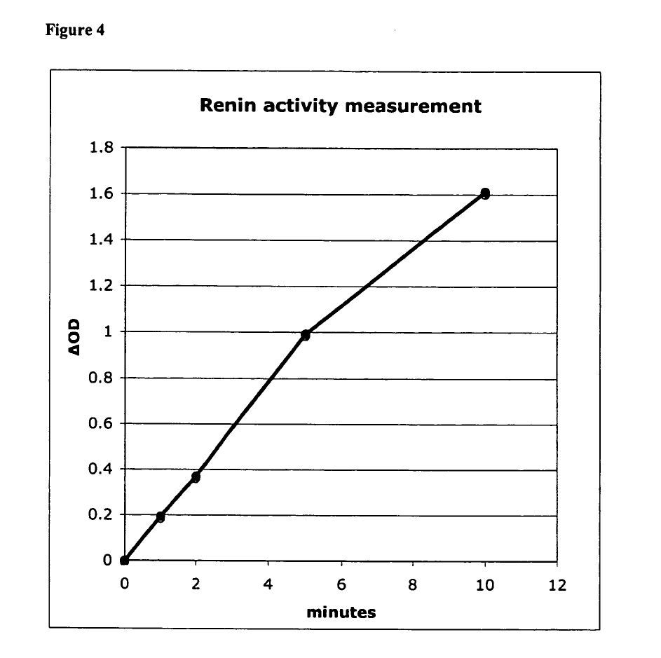

FIG. 4: Difference in Optical Density to the empty control solution after incubation of the Renin enzyme solution with the embodiment of the invention and the development of the signal, as described below.

FIG. 5: FIG. 5 shows a longitudinal cross section of a first embodiment of the reaction device according to the invention;

FIGS. 6-9: FIGS. 6, 7, 8 and 9 show a second, a third, a fourth and a fifths embodiment of the reaction device according to the invention, respectively.

### EXAMPLES

### Example 1

In this example the enzyme pepsin, an aspartic protease, from porcine gastric juice was tested according to the method of the invention. The test was illustrated by using first the chromophoric peptide substrate H-Pro-Thr-Glu-Phe-(NO₂-Phe)-Arg-Leu-OH (Bachem Pr.Nr.: H-1002) according to the available specified method (Dunn B M, Kammermann B, and Mc Curry H R. Anal Biochem 1984; 138 (1): 68-73) The reaction was monitored at 310 nm, at which wave length a difference between the absorbance of the substrate and the product was detected.

The same substrate was then embedded according to the method of the invention and reacted with the enzyme porcine pepsin. The produced signal(s), using the same substrate, were compared (see FIG. 3):
A. Preparation of the substrate, bound to a Sepharose fast flow gel as anchorage entity A and the peptide H-Pro-Thr-Glu-Phe-(NO₂-Phe)-Arg-Leu-OH (Bachem Pr.Nr.:H-1002) as substrate S:
   1.5 g activated insoluble anchorage entity A (Sepharose) were bound covalently to a linker L1, a spacer arm with a length of 20 C atoms, to yield A-L1.
   2. The linker L1 in A-L1 was then activated and bound to 12.5 mg of substrate S (H-Pro-Thr-Gluc-Phe-(NO₂-Phe)-Arg-Leu-OH) to yield A-L1-S.
   3. Excess of activated insoluble anchorage entity positions in A-L1, were blocked with Tris buffer 0.1 M, pH 8.0.
   4. Activation of the linked substrate S in A-L1-S and binding to a second linker L2 with a length of 20 C atoms, were performed through methods known to the skilled person.
   5. Activation of the free linker L2 in A-L1-S-L2 and binding to the marker M, which consisted of HRP (Horse Radish Peroxidase) Type II (Sigma Pr.Nr.: P 8250), 100,000 Units (400 mg) resulted in the embodiment A-L1-S-L2-M. Excess of free activated positions on the embodiment A-L1-S-L2 were blocked with Tris buffer 0.1 M, pH 8.0.
The product was then washed with the same buffer at least twice and stored at 4° C.
B. The reaction of the enzyme pepsin with the chromophoric peptide substrate as such: A pepsin dilution series containing 1 and 10 μg of Pepsin (Sigma Pr.Nr.: P-6887; 3,200 units/mg solid) in 1 ml 0.1 M tri-Sodium Citrate Dihydrate (Fluka Pr.Nr.: 71403), 0.1 M Sodium Chloride (Fluka Pr.Nr.: 71381) pH 3.5, was prepared.
   1. 12.5 mg of the Pepsin substrate (Bachem Pr.Nr.: H-1002) was diluted in 2.5 ml 10 mM tri-Sodium Citrate Dihydrate (Fluka Pr.Nr.: 71403), 10 mM Sodium Chloride (Fluka Pr.Nr.: 71381) pH 3.5.

2. 50 μl of the Pepsin substrate solution were added to 850 μl reaction buffer solution. Reaction buffer: 0.1 M tri-Sodium Citrate Dihydrate (Fluka Pr.Nr.: 71403), 0.1 M Sodium Chloride (Fluka Pr.Nr.: 71381) pH 3.5 and placed in an Amersham Ultrospec 2000 spectrophotometer.
3. 100 μl of the Pepsin enzyme solution was added to the Pepsin substrate solution and reaction buffer. The decrease in optical density was measured at 310 nm and room temperature over a time period of 30 minutes.

### Results (see FIG. 3):

1 μg/ml Pepsin [symbol] ΔOD 310 nm=0.019
10 μg/ml Pepsin [symbol] ΔOD 310 nm=0.116
C. The reaction of Pepsin with A-L1-S-L2-M prepared as described above in chapter A.
   1. A Pepsin dilution series containing 1 and 10 μg of Pepsin (Sigma Pr.Nr.: P-6887, 3,200 units/mg solid) in 1 ml 0.5 M MES (Sigma Pr.Nr.: M2933), 0.5 M Sodium Chloride (Fluka Pr.Nr.: 71381), 50 mM CaCl₂, pH 3.5 was prepared.
   2. 100 μl of the Pepsin enzyme solution were added to 50 mg A-L1-S-L2-M and incubated at 21° C. over a time period of 15 minutes.
   3. The Pepsin enzyme solution was separated from remaining A-L1-S-L2-M on a Millipore Microcon YM-100 centrifugal filter device with cut off 100,000 MW; after centrifugation for 2 minutes at a speed of 14,500 rpm.
   4. 100 μl of the solution containing split A-L1-S-L2-M was added to 900 μl 2,2-Azino-Bis(3-Ethylbenzthiazoline-6-Sulfonic Acid) liquid horse radish perioxidase type II substrate solution (Sigma Pr.Nr.: A 3219). The increase in optical density was measured at 405 nm and room temperature over a time period of 30 minutes, in an Amersham Ultrospec 2000 spectrophotometer containing a plastic cell.

### Results (see FIG. 3):

1 μg/ml Pepsin [symbol] ΔOD 405 nm=0.304
10 μg/ml Pepsin [symbol] ΔOD 405 nm=0.784

### Example 2

In this example the enzyme renin from human plasma is tested according to the method of the invention. The test is illustrated by using the peptide substrate H-Asp-Arg-Val-Tyr-Ile-His-Pro-Phe-His-Leu-Val-Ile-His-Ser-OH.

The substrate was embedded according to the method of the invention and reacted with the enzyme human plasma renin. The produced signal is shown (see FIG. 4):
A. Preparation of the substrate, bound to a Sepharose fast flow gel as anchorage entity A and the peptide H-Asp-Arg-Val-Tyr-Ile-His-Pro-Phe-His-Leu-Val-Ile-His-Ser-OH (Bachem Pr. Nr.: M2500) as substrate S:
   1. 2.2 g activated insoluble anchorage entity A (Sepharose) were bound covalently to a linker L1, a spacer arm with a length of 20 C atoms, to yield A-L1.
   2. The linker L1 in A-L1 was then activated and bound to 5 mg of substrate S(H-Asp-Arg-Val-Tyr-Ile-His-Pro-Phe-His-Leu-Val-Ile-His-Ser-OH) to yield A-L1-S.
   3. Excess of activated insoluble anchorage entity positions in A-L1, was blocked with Tris buffer 0.1 M, pH 8.0.
   4. Activation of the linked substrate S in A-L1-S and binding to a second linker L2 with a length of 20 C atoms, by methods known to the skilled person.
   5. Activation of the free linker L2 in A-L1-S-L2 and binding to the marker M, which consists of HRP (Horse Radish Peroxidase) Type II (see above), 100,000 Units (400 mg) resulting in A-L1-S-L2-M. Excess of free activated positions on A-L1-S-L2 were blocked with Tris buffer 0.1 M, pH 8.0. The concentration of the substrate complex was then diluted 50× by addition of sepharose fast flow gel (Amersharn Pr. Nr.: 17-0120-01). Thereafter, the complex was washed with MES buffer (0.1 M MES (see above), 0.5 M NaCl (Fluka Pr. Nr.: 71381), 0.05 M CaCl₂ (Fluka Pr. Nr.: 21097), 0.01% Thimerosal (Sigma Pr. Nr.: T8784), pH 7.0) at least twice and stored at 4° C.
B. The reaction of Renin with A-L1-S-L2-M prepared as described above in chapter A.
   1. 0.1 mg partially purified human plasma Renin (Bio Pur P. Nr.: 10-13-1121), containing 0.7 ng active Renin was dissolved in 2 ml H₂O.
   2. 2 ml of the Renin enzyme solution were added to 1.2 g A-L1-S-L2-M dissolved in 2 ml MES buffer pH 7 and incubated at 21° C. over a time period of 15 minutes.
   3. In a second preparation 2 ml of MES buffer pH 7 were added to 1.2 g A-L1-S-L2-M dissolved in 2 ml MES buffer pH 7 and incubated at 21° C. over a time period of 15 minutes, for reference.
   4. The preparation solutions were separated by filtration.
   5. 100 μl of the solution containing the split A-L1-S-L2-M was added to 900 μl 2,2-Azino-Bis(3-Ethylbenzthiazoline-6-Sulfonic Acid) liquid horse radish perioxidase type II substrate solution (see above). The increase in optical density was measured at 405 nm and room temperature over a time period of 10 minutes, in an Amersham Ultrospec 2000 spectrophotometer containing a plastic cell.

Results (see FIG. 4):
1 minute Renin signal development [symbol] ΔOD 405 nm=0.193
2 minutes Renin signal development [symbol] ΔOD 405 nm=0.369
5 minutes Renin signal development [symbol] ΔOD 405 nm=0.993
10 minutes Renin signal development [symbol] ΔOD 405 nm=1.612

### Example 3

### Especially Preferred Kits of the Invention and Methods for Using them

The following kits 1-8 may optionally further contain appropriate buffer conditions, which the skilled person will be able to determine. Furthermore, the skilled person will appreciate that other combinations of the kit components indicated above are also possible.

### Kit 1: for the Detection of Pepsin

### A: Sepharose

L1 and L2, respectively: linker molecules of the size C20
S: substrate H-Pro-Thr-Glu-Phe-(NO₂-Phe)-Arg-Leu-OH
M: enzyme HRP

### Kit 2: for the Detection of Renin

A: a nitrocellulose surface
L1 and L2, respectively: linker molecules of the size C22
S: substrate H-Asp-Arg-Val-Tyr-Ile-His-Pro-Phe-His-Leu-Val-Ile-His-OH
M: soluble dye azorubin

### Kit 3: for the Detection of Cathepsin D

R: sepharose-bound streptavidin
A: biotin
L1 and L2, respectively: linker molecules of the size C18
S: substrate H-Asp-Arg-Val-Tyr-Ile-His-Pro-Phe-His-Leu-Leu-Val-Tyr-Ser-OH
M: enzyme β-galactosidase.
Kit 4: for the detection of T-Cell Leukemia Virus Type I-Protease
A: blue dextran with a molecular size of 200 kDa
B: L1 and L2, respectively: linker molecules of the size C25
S: substrate H-Ala-Pro-Gln-Val-Leu-Phe-Val-Met-His-Pro-Leu-OH
M: a chemical compound containing a free thiol group

### Kit 5: for the Detection of Secretase

A: blue dextran with a molecular size of 2000 kDa
L1 ad L2, respectively: linker molecules of the size C30
S: substrate H—Ser-Glu-Val-Asn-Leu-Asp-Ala-Glu-Phe-OH
M: enzyme acetylcholine-esterase

### Kit 6: for the Detection of Thrombin

R: a Nickel containing surface

### A: a His-tag-fusion-protein

L1 and L2, respectively: linker molecules of the size C20
S: substrate H-Phe-Pro-Arg-OH
M: enzyme alkaline phosphatase.

### Kit 7: for the Simultaneous Detection of Kallikrein, Renin and Thrombin

R: a magnetic surface
A: Fe-ions containing surface
L1 and L2, respectively: linker molecules of the size C28
S1: Kallikrein substrate H-D-Pro-Phe-Arg-OH
S2: Renin substrate H-Asp-Arg-Val-Tyr-Ile-His-Pro-Phe-His-Leu-Val-Ile-His-OH
S3: Thrombin substrate H-Phe-Pro-Arg-OH
M1: a molecular tag of the size 3000 Da
M2: a molecular tag of the size 5000 Da
M3: is a molecular tag of the size 10'000 Da

### Kit 8: for the Detection of Multiple Renin Substrates

A: a glass surface
L1 and L2, respectively: linker molecules of the size C30
S1: substrate H-Asp-Arg-Val-Tyr-Ile-His-Pro-Phe-His-Leu-Val-Ile-His-OH
S2: substrate H-Asp-Arg-Val-Tyr-Ile-His-Pro-Phe-His-Leu-Val-Ile-His-Asn-OH
S3: substrate H-Asp-Arg-Val-Tyr-Ile-His-Pro-Phe-His-Leu-Val-Ile-His-Ser-OH
S4: substrate H-Asp-Arg-Val-Tyr-Ile-His-Pro-Phe-His-Leu-Leu-Val-Tyr-Ser-OH
M1: enzyme horse radish peroxidase
M2: enzyme alkaline phosphatase
M3: enzyme β-galactosidase
M4: enzyme acetylcholine-esterase

### Kit 9 Enzyme Coupled Substrates Kit for the Detection of Multiple Enzymes

This kit is composed of 9 components which are:
1. An insoluble removable entity R, e.g, Streptavidin or Avidin covalently coupled to e.g. sepharose
   Component 1 is contained in a test tube, with an appropriate buffer.
2. A soluble substrate complex A-L1-S-L2-E2 with A being e.g.: Biotin and L1 and L2 being a linker molecule, e.g. an alkane of the length C20 and with S being e.g.:
   H-Gly-Lys-OH, H-Pro-Arg-OH, H-Val-Arg-OH, H-Val-Pro-Arg-OH, H-Phe-Val-Arg-OH, H-Phe-Arg-OH, H-Phe-Pro-Arg-OH, H-Gly-Pro-Lys-OH, H-Gly-Gly-Arg-OH, H-Gly-Pro-Arg-OH and any derivatives of these for Coagulation Factor IIa (Thrombin).
   H-Asp-Arg-Val-Tyr-Ile-His-Pro-Phe-His-Leu-Leu-Val-Tyr-Ser-OH, H-Asp-Arg-Val-Tyr-Ile-His-Pro-Phe-His-Leu-Val-Ile-His-Asn-OH, H-Asp-Arg-Val-Tyr-Ile-His-Pro-Phe-His-Leu-Leu-Tyr-Tyr-Ser-OH, H-Asp-Arg-Val-Tyr-Ile-His-Pro-Phe-His-Leu-Leu-Val-Tyr-Ser-OH, H-Asp-Arg-Val-Tyr-Ile-His-Pro-Phe-His-Leu-Val-Ile-His-OH, H-Asp-Arg-Val-Tyr-Ile-His-Pro-Phe-His-Leu-Val-Ile-His-Asn-OH, H-Asp-Arg-Val-Tyr-Ile-His-Pro-Phe-His-Leu-Val-Ile-His-Ser-OH, H-Ile-His-Pro-Phe-His-Leu-Val-Ile-His-Thr-OH, H-Arg-Pro-Phe-His-Leu-Leu-Val-Val-Tyr-OH, H-Pro-Phe-His-Leu-Leu-Val-Tyr-Ser-OH
   and any derivatives of these for Renin.
   H-Glu-Gly-Arg-OH and any derivatives of it for Coagulation Factor IXa.
   H-Ile-Glu-Gly-Arg-OH, H-Leu-Gly-Arg-OH, H-Gly-Pro-Lys-OH and any derivatives of these for Coagulation Factor Xa.
   H-Glu-Ala-Arg-OH, H-Phe-Ser-Arg-OH, H-Pyr-Pro-Arg-OH and any derivatives of these for Coagulation Factor XIa.
   H-Phe-Arg-OH, H-Gln-Gly-Arg-OH, H-Glu-Gly-Arg-OH, H-Ile-Glu-Gly-Arg-OH and any derivatives of these for Coagulation Factor XIIa.
   H-Met-Leu-Ala-Arg-Arg-Lys-Pro-Val-Leu-Pro-Ala-Leu-Thr-Ile-Asn-Pro-OH and any derivatives of it for Anthrax Lethal Factor.
   H-Asp-Glu-Val-Asp-OH, H-Asp-Met-Gln-Asp-OH, H-Asp-Glu-Val-Asp-Ala-Pro-Lys-OH, H-Asp-Gln-Met-Asp-OH and any derivatives of these for Casapase-3.
   H-Glu-Asp-Lys-Pro-Ile-Leu-Phe-Phe-Arg-Leu-Gly-Lys-Glu-OH, H-Asp-Arg-Val-Tyr-Ile-His-Pro-Phe-His-Leu-Leu-Val-Tyr-Ser-OH, H-Arg-Gly-Phe-Phe-Leu-OH, H-Arg-Gly-Phe-Phe-Pro-OH, H-Gly-Lys-Pro-Ile-Leu-Phe-Phe-Arg-Leu-Lys-Arg-OH, H-Phe-Ala-Ala-Phe-Phe-Val-Leu-OH, H-Phe-Gly-His-Phe-Phe-Ala-Phe-OH, H-Phe-Ser-Phe-Phe-Ala-Ala-OH, H-Pro-Thr-Glu-Phe-Phe-Arg-Leu-OH and any derivatives of these for Cathepsin D.
   H-Nal-Abu-Phe-Abu-Abu-Nal-OH and any derivatives of it for Feline Immunodeficiency Virus (FIV) protease.
   H-Asp-Glu-Asp-Glu-Glu-Abu-Ser-Lys-OH, H-Glu-Ala-Gly-Asp-Asp-Ile-Val-Pro-Cys-Ser-Met-Ser-Tyr-Thr-Trp-Thr-Gly-Ala-OH and any derivatives of these for Hepatitis C Virus (HCV) NS3 protease.
   H-Arg-Gly-Val-Val-Asn-Ala-Ser-Ser-Arg-Leu-Ala-Lys-OH, H-Arg-Gly-Val-Val-Asn-Ala-Ser-Ser-Arg-Leu-Ala-OH and any derivatives of these for Human Cytomegalovirus (CMV) protease (Assemblin).
   H-Ala-Pro-Gln-Val-Leu-Phe-Val-Met-His-Pro-Leu-OH and any derivatives of it for Human T-Cell Leukemia Virus Type I (HTLV-I) prtotease.
   H-Phe-Arg-OH, H-Ile-Glu-Gly-Arg-OH, H-Pro-Phe-Arg-OH, H-Val-Leu-Arg-OH and any derivatives of these for Kallikrein.
   H-Val-Ser-Val-Asn-Ser-Thr-Leu-Gln-Ser-Gly-Leu-Arg-Lys-Met-Ala-OH and any derivatives of it for SARS protease.
   H-Ala-Ala-Pro-Phe-OH, H-Ala-Ala-Phe-OH, H-Gly-Gly-Phe-OH, H-Ala-Ala-Pro-Met-OH, H-Ala-Ile-Pro-Met-OH, H-Ser-Glu-Val-Asn-Leu-Asp-Ala-Glu-Phe-OH, H-Phe-Leu-Phe-OH, H-Val-Pro-Phe-OH and any derivatives of these for Chymotrypsin.
   H-Gln-Ala-Arg-OH, H-Gln-Gly-Arg-OH, H-Val-Gly-Arg-OH, H-Ala-Ala-Pro-Arg-OH, H-Gly-Gly-Arg-OH, H-Ala-Ala-Pro-Lys-OH, H-Glu-Gly-Arg-OH and any derivatives of these for Trypsin.

H-Gly-Gly-Phe-Phe-OH, H-Leu-Ser-Phe-Nle-Ala-Leu-OH, H-Phe-Ala-Ala-Phe-Phe-Val-Leu-OH, H-Phe-Gly-His-Phe-Phe-Ala-Phe-OH, H-Pro-Thr-Glu-Phe-Phe-Arg-Leu-OH, H-His-Phe-Phe-OH, H-His-Phe-Trp-OH, H-His-Phe-Tyr-OH, H-His-Tyr-Tyr-OH and any derivatives of these for Pepsin.
and with marker enzyme E2 being e.g. horse radish peroxidase.
Component 2 is contained in a test tube, with an appropriate buffer.
3. A reference enzyme contained in a test tube in an appropriate buffer; for example:
   Coagulation Factor IIa, Renin, Coagulation Factor IXa, Coagulation Factor Xa, Coagulation Factor Xla, Coagulation Factor XIIa, Anthrax Lethal Factor, Caspase-3, Cathepsin D, Feline Immunodeficiency Virus (FIV) protease, Hepatitis C Virus (HCV) NS3 protease, Human Cytomegalovirus (CMV) protease (Assemblin), Human T-Cell Leukemia Virus Type I (HTLV-I) prtotease, Kallikrein, SARS protease, Chymotrypsin, Trypsin, Pepsin.
4. A substrate solution for E2, e.g. a peroxidase substrate, contained in test tube with the appropriate buffer.
5. Plastic cuvettes for the measurement of the enzyme reaction, e.g. peroxidase reaction at 410 mm in an appropriate spectrophotometer.
6. Additional test tubes.
7. Several centrifugal filter devices with cut-off MW 100,000 Da for use in a test tube centrifuge.
8. A 1% SDS solution to stop the E2-reaction at the appropriate time point.
9. Control Buffer, identical to the buffer used in component 3.

### Procedure for the use of Kit 9:

1. Incubate the sample containing the target enzyme E1, component 3 containing the reference enzyme for E1 or component 9 containing the empty buffer control, with a sample of the component 2, for 15 minutes reaction in component 6.
2. Add, for example, 50 mg of component 1 and incubate for an additional 15 minutes in component 6.
3. Filtrate the mixture through component 7.
4. Add component 4 to component 5 in an appropriate spectrophotometer.
5. Add the filtrated mixture of step 3 to component 5 containing component 4 and reset the measurement of the spectrophotometer.
6. Add component 8 after 30 minutes to stop the reaction of E2 with S2 and measure the signal.

The advantage of this kit is the possibility to enhance the enzymatic activity of enzymes contained in trace amounts in a sample enabling a quick and easy detection.

### Kit 10 Chemical Tagged Substrates Kit for the Detection of Enzymes

The kit is composed out of 4 components, which are:
1. Component 1: a substrate complex A-L1-S-L2-M, with A being a plastic, polyacrylic, ceramic or other unsoluble membrane surface comprising the bottom or the walls of a corresponding cuvette, L1 and L2 being linker molecules, e.g. an alkane of a length of C20 and S being a substrate, e.g.
   H-Gly-Lys-OH, H-Pro-Arg-OH, H-Val-Arg-OH, H-Val-Pro-Arg-OH, H-Phe-Val-Arg-OH, H-Phe-Arg-OH, H-Phe-Pro-Arg-OH, H-Gly-Pro-Lys-OH, H-Gly-Gly-Arg-OH, H-Gly-Pro-Arg-OH and any derivatives of these for Coagulation Factor IIa (Thrombin).
   H-Asp-Arg-Val-Tyr-Ile-His-Pro-Phe-His-Leu-Leu-Val-Tyr-Ser-OH, H-Asp-Arg-Val-Tyr-Ile-His-Pro-Phe-His-Leu-Val-Ile-His-Asn-OH, H-Asp-Arg-Val-Tyr-Ile-His-Pro-Phe-His-Leu-Leu-Tyr-Tyr-Ser-OH, H-Asp-Arg-Val-Tyr-Ile-His-Pro-Phe-His-Leu-Leu-Val-Tyr-Ser-OH, H-Asp-Arg-Val-Tyr-Ile-His-Pro-Phe-His-Leu-Val-Ile-His-OH, H-Asp-Arg-Val-Tyr-Ile-His-Pro-Phe-His-Leu-Val-Ile-His-Asn-OH, H-Asp-Arg-Val-Tyr-Ile-His-Pro-Phe-His-Leu-Val-Ile-His-Ser-OH, H-Ile-His-Pro-Phe-His-Leu-Val-Ile-His-Thr-OH, H-Arg-Pro-Phe-His-Leu-Leu-Val-Val-Tyr-OH, H-Pro-Phe-His-Leu-Leu-Val-Tyr-Ser-OH and any derivatives of these for Renin.
   H-Glu-Gly-Arg-OH and any derivatives of it for Coagulation Factor IXa. H-Ile-Glu-Gly-Arg-OH, H-Leu-Gly-Arg-OH, H-Gly-Pro-Lys-OH and any derivatives of these for Coagulation Factor Xa.
   H-Glu-Ala-Arg-OH, H-Phe-Ser-Arg-OH, H-Pyr-Pro-Arg-OH and any derivatives of these for Coagulation Factor XIa.
   H-Phe-Arg-OH, H-Gln-Gly-Arg-OH, H-Glu-Gly-Arg-OH, H-Ile-Glu-Gly-Arg-OH and any derivatives of these for Coagulation Factor XIIa.
   H-Met-Leu-Ala-Arg-Arg-Lys-Pro-Val-Leu-Pro-Ala-Leu-Thr-Ile-Asn-Pro-OH and any derivatives of it for Anthrax Lethal Factor.
   H-Asp-Glu-Val-Asp-OH, H-Asp-Met-Gln-Asp-OH, H-Asp-Glu-Val-Asp-Ala-Pro-Lys-OH, H-Asp-Gln-Met-Asp-OH and any derivatives of these for Casapase-3.
   H-Glu-Asp-Lys-Pro-Ile-Leu-Phe-Phe-Arg-Leu-Gly-Lys-Glu-OH, H-Asp-Arg-Val-Tyr-Ile-His-Pro-Phe-His-Leu-Leu-Val-Tyr-Ser-OH, H-Arg-Gly-Phe-Phe-Leu-OH, H-Arg-Gly-Phe-Phe-Pro-OH, H-Gly-Lys-Pro-Ile-Leu-Phe-Phe-Arg-Leu-Lys-Arg-OH, H-Phe-Ala-Ala-Phe-Phe-Val-Leu-OH, H-Phe-Gly-His-Phe-Phe-Ala-Phe-OH, H-Phe-Ser-Phe-Phe-Ala-Ala-OH, H-Pro-Thr-Glu-Phe-Phe-Arg-Leu-OH and any derivatives of these for Cathepsin D. H-Nal-Abu-Phe-Abu-Abu-Nal-OH and any derivatives of it for Feline Immunodeficiency Virus (FIV) protease.
   H-Asp-Glu-Asp-Glu-Glu-Abu-Ser-Lys-OH, H-Glu-Ala-Gly-Asp-Asp-Ile-Val-Pro-Cys-Ser-Met-Ser-Tyr-Thr-Trp-Thr-Gly-Ala-OH and any derivatives of these for Hepatitis C Virus (HCV) NS3 protease.
   H-Arg-Gly-Val-Val-Asn-Ala-Ser-Ser-Arg-Leu-Ala-Lys-OH, H-Arg-Gly-Val-Val-Asn-Ala-Ser-Ser-Arg-Leu-Ala-OH and any derivatives of these for Human Cytomegalovirus (CMV) protease (Assemblin).
   H-Ala-Pro-Gln-Val-Leu-Phe-Val-Met-His-Pro-Leu-OH and any derivatives of it for Human T-Cell Leukemia Virus Type I (HTLV-I) prtotease.
   H-Phe-Arg-OH, H-Ile-Glu-Gly-Arg-OH, H-Pro-Phe-Arg-OH, H-Val-Leu-Arg-OH and any derivatives of these for Kallikrein.
   H-Val-Ser-Val-Asn-Ser-Thr-Leu-Gln-Ser-Gly-Leu-Arg-Lys-Met-Ala-OH and any derivatives of it for SARS protease.
   H-Ala-Ala-Pro-Phe-OH, H-Ala-Ala-Phe-OH, H-Gly-Gly-Phe-OH, H-Ala-Ala-Pro-Met-OH, H-Ala-Ile-Pro-Met-OH, H-Ser-Glu-Val-Asn-Leu-Asp-Ala-Glu-Phe-OH, H-Phe-Leu-Phe-OH, H-Val-Pro-Phe-OH and any derivatives of these for Chymotrypsin.
   H-Gln-Ala-Arg-OH, H-Gln-Gly-Arg-OH, H-Val-Gly-Arg-OH, H-Ala-Ala-Pro-Arg-OH, H-Gly-Gly-Arg-OH, H-Ala-Ala-Pro-Lys-OH, H-Glu-Gly-Arg-OH and any derivatives of these for Trypsin.
   H-Gly-Gly-Phe-Phe-OH, H-Leu-Ser-Phe-Nle-Ala-Leu-OH, H-Phe-Ala-Ala-Phe-Phe-Val-Leu-OH, H-Phe-Gly-His-Phe-Phe-Ala-Phe-OH, H-Pro-Thr-Glu-Phe-Phe-Arg-Leu-OH, H-His-Phe-Phe-OH, H-His-Phe-Trp-OH, H-His-Phe-Tyr-OH, H-His-Tyr-Tyr-OH and any derivatives of these for Pepsin.
   M being a marker dye, e.g., Phthalocyanine, diazonium, diphenylmethane, anthraquinone, acridine, quinone-imine, eurrhodin, safranin, oxazin, oxazone, thiazin, thiazole, xanthene, pyronin, rhodamine, fluorine or other dye molecules.
2. Component 2: an appropriate buffer for component 1.
3. Component 3: a reference for enzyme E1 contained in a test tube, with an appropriate buffer, for example: Coagulation Factor IIa, Renin, Coagulation Factor Ixa, Coagulation Factor Xa, Coagulation Factor XIa, Coagulation Factor XIIa, Anthrax Lethal Factor, Caspase-3, Cathepsin D, Feline Immunodeficiency Virus (FIV) protease, Hepatitis C Virus (HCV) NS3 protease, Human Cytomegalovirus (CMV) protease (Assemblin), Human T-Cell Leukemia Virus Type I (HTLV-I) prtotease, Kallikrein, SARS protease, Chymotrypsin, Trypsin, Pepsin.
4. Component 4: Reference buffer, identical to the buffer used in component 2.
   Procedure for using Kit 10
1. Incubate component 1 with a sample of the targeted enzyme E1 and component 2, the corresponding buffer, for 15 or 30 minutes as described.
2. Incubate the reference component 3 with the corresponding reference buffer 4 for 15 or 30 minutes as described.
3. In an appropriate spectrophotometer set the appropriate wavelength as described for measuring the concentration of the dye substance in solution.
4. Stop the reaction of E1 by adding 1% SDS solution to the sample and add the same to the reference solution.
5. Shake the reaction and the reference solutions.
6. Start the spectrophotometric measurement.
7. Write down the measured reference signal and the test signal after 15 or 30 minutes and deduce the corresponding E1 concentration as described.

The advantage of this product is the ability to measure trace amounts of an enzyme in sample solutions, due to the high signal intensity of the used dye, and the short and simple test procedures.

1. A method for the detection of an enzyme E1 in a liquid sample comprising the steps of
a) providing a complex (Sa-Sb-M), wherein (Sa-Sb) is a substrate S of E1 cleavable into Sa and Sb by E1, and M is a marker linked to Sb,
b) incubating the sample with the complex under conditions enabling the cleavage of S into Sa and Sb by E1, thereby generating complex Sb-M,
c) separating non-cleaved complex (Sa-Sb-M) from complex Sb-M, and
d) measuring M in the sample
wherein the separating of step c) does not involve a magnetic field.

2. The method of claim 1, wherein the complex Sb-M is released into the liquid phase as a result of the cleavage of step b).

3. The method of claim 2, wherein the complex Sa-Sb-M is immobilized during steps a) to c) and optionally d).

4. The method of claim 1, wherein the complex Sa-Sb-M provided in step a) is bound to a surface of a reaction chamber in which the reaction takes place.

5. (canceled)

6. The method of claim 1, wherein the steps b) and d) are performed at distinct sections or distinct positions in the same reaction chamber.

7. The method of claim 1, wherein the M comprises an enzyme E2.

8. The method of claim 7, wherein E2 is selected from the group consisting of a peroxidase, a phosphatase, a luciferase, a monooxygenase, horse radish peroxidase (HRP), soybean peroxidase, alkaline phosphatase (AKP), acidic phosphatase, photinus-luciferin 4-monooxygenase, renilla-luciferin 2-monooxygenase, cypri-dinialuciferin 2-monooxygenase, watasenia-luciferin 2-monooxygenase, oplophorus-luciferin 2-monooxygenase, beta-galactosidase, and acetyl cholin-esterase.

9.-12. (canceled)

13. The method of claim 1, wherein E1 is selected from the group consisting of a hydrolytic enzyme, a phosphorolytic enzyme, a peptide hydrolase, lipase, glycosylase, nuclease, and other hydrolase.

14.-16. (canceled)

17. The method of claim 1, wherein Sa is further linked to an anchor entity A, such that after the cleavage in step b) at least the complexes (Sa-A) and (Sb-M) are formed.

18.-20. (canceled)

21. The method of claim 17, wherein A is a substance selected from a group consisting of a high molecular soluble compound and a part of an insoluble matrix.

22. The method of claim 21, wherein the substance is selected from the group consisting of a compound with a molecular weight of 100 kDa or higher, a dextran, protein, gelatine, polyglycan, polyxylan, amylase, amylopectin, galactan, polynucleic acid, a dye, a substance comprising a dye, a sepharose, cellulose, sephadex, silica gel, acrylic bed or other resin, ceramic bed, Wafer glass, amorphous silicon carabide, castable oxidus, polyimides, polymethylmethacrylates, polystyrenes, gold or silicone elastomers, and nitrocellulose.

23.-26. (canceled)

27. The method ofclaim 1, wherein non-cleaved S, but not the complex (Sb-M), is linked to a removable entity R after step b).

28. The method of claim 27, wherein said linking is effected by linking R to an anchor entity A linked to Sa such that after the cleavage in step b) at least the complexes (Sa-A) and (Sb-M) are formed.

29.-31. (canceled)

32. The method of claim 28, wherein the non-cleaved complex is separated from the sample by removing the A-R complex.

33. (canceled)

34. The method of claim 1, wherein M is a chemical compound.

35. The method of claim 34, wherein the chemical compound is selected from the group consisting of a dye substance, chromophore, fluoromere, a molecular tag with a molecular weight of least 100 Da, an organic molecule with a functional group such as alcohol, aldehyde, amine, dibromoamine, thoil, a pH dye indicator such as phenolphthalein (3,3-Bis(4-hydroxyphenyl)-1(3H)-isobenzofuranone), and glucose.

36.-40. (canceled)

41. The method of claim 1, wherein two or more complexes (Sa-Sb-M) with different substrates S for different enzymes E1 and different markers M are provided, thereby enabling the detection of these E1.

42. The method of claim 1, wherein two or more complexes (Sa-Sb-M) with different substrates S for one enzyme E1 and different markers M are provided, thereby enabling the testing the reaction of E1 with multiple substrates in a sample.

43.-45. (canceled)

46. A reaction device for the detection of an enzyme E1 in a liquid sample, the reaction device comprising:
a reaction chamber;
a first surface, the first surface covering a continuous area or a plurality of continuous areas being mutually connected;
a second surface;
a complex Sa-Sb-M, essentially the complete amount of Sa-Sb-M comprised by the reaction device being bound on the first surface, wherein Sa-Sb is a substrate S of E1 cleavable into Sa and Sb by E1, M is a marker linked to Sb, and M comprises an enzyme E2; and
a substrate S2 being located on the second surface, wherein substrate S2 is a substrate of E2, wherein cleavage of S2 by E2 generates a signal, wherein the first surface is distinct from the second surface;
the reaction device further comprising a separation assembly spatially separating the marker M linked with uncleaved substrate S from substrate S2, the separation assembly being connected to the first surface.

47. The reaction device of claim 46, the separation assembly further comprising a bond between substrate S2 and the second surface.

48.-49. (canceled)

50. The reaction device of claim 46, the separation assembly further comprising an actuation assembly having a first element connected to the first surface as well as a second element connected to the second surface, the actuation assembly being adapted to remove the first surface from the liquid sample by means of the first element, and to establish direct contact between the second surface and the liquid sample as well as to substrate S2 by means of the second element, the first element and the second element being connected by a mechanical or electrical connection adapted to establish the direct contact exclusively after the complete removal of the first surface.

51.-52. (canceled)

53. The reaction device of claim 46, further comprising a first support and a second support, the first surface being located on the first support and the second surface being located on the second support, wherein the reaction chamber is adapted to receive the liquid sample and the first and the second support, the reaction chamber being adapted to receive only one of the first and the second support at a time or being adapted to receive both, the first and the second support, simultaneously, the at least one of the first and the second support being configured to be partly or completely immersed into the liquid sample.

54. (canceled)

55. A reaction device for the detection of an enzyme E1 in a liquid sample, the reaction device comprising:
a carrier having a first surface section and a second surface section;
a complex Sa-Sb-M, essentially the complete amount of Sa-Sb-M comprised by the reaction device being bound on the first surface, wherein Sa-Sb is a substrate S of E1 cleavable into Sa and Sb by E1, M is a marker linked to Sb, and M comprises an enzyme E2; and
a substrate S2, essentially the complete amount being bound on the second surface, wherein substrate S2 is a substrate of E2, and cleavage of S2 by E2 generates a signal;
the first surface section being separated from the second surface section.

56.-57. (canceled)

58. An array of reaction devices according to claim 46, each reaction device being dedicated to a distinct one of a plurality of liquid samples, each array comprising a complex Sa-Sb-M being specific to the same enzyme E1 wherein Sa-Sb is a substrate S of E1 cleavable into Sa and Sb by E1, or, alternatively, each array comprising a distinct complex Sa-Sb-M, each being specific to one of a plurality of distinct enzymes E1n, wherein M is a marker linked to Sb and comprises an enzyme E2; the array further comprising a plurality of substrates S2, each substrate S2 being specific to the enzyme E2, and cleavage of the plurality of S2 by E2 generating a plurality of specific signals SIGn, each signal SIGn being related to a specific reaction device comprised by the array, wherein each of the specific signals SIGn has a distinct location of occurrence, the location of occurrences comprising surfaces of distinct reaction devices or volumes of distinct liquid samples.

59. An array of reaction devices according to claim 46, the array comprising a plurality of complexes Sa-Sb-Mn, each being specific to one of a plurality of distinct enzymes E1n, wherein Sa-Sb is a substrate Sn of E1n cleavable into Sa and Sb by E1n,
wherein Mn is a marker linked to Sb of each of the complexes Sa-Sb-Mn, and Mn comprises an enzyme E2n; the array further comprising a plurality of substrates S2n, each substrate S2n being specific to one enzyme E2n, wherein cleavage of each S2n by E2n generates at least one of a plurality of distinct signals SIGn, whereby the signals SIGn are mutually distinguishable.


SEQUENCE LISTING (46 sequences)

SEQ ID NO: 1 - protein, 14 aa, artificial sequence
  substrate for Renin
DRVYIHPFHLLVYS

SEQ ID NO: 2 - protein, 14 aa, artificial sequence
  substrate for Renin
DRVYIHPFHLVIHN

SEQ ID NO: 3 - protein, 14 aa, artificial sequence
  substrate for Renin
DRVYIHPFHLLYYS

SEQ ID NO: 4 - protein, 14 aa, artificial sequence
  substrate for Renin
DRVYIHPFHLLVYS

SEQ ID NO: 5 - protein, 13 aa, artificial sequence
  substrate for Renin
DRVYIHPFHLVIH

SEQ ID NO: 6 - protein, 14 aa, artificial sequence
  substrate for Renin
DRVYIHPFHLVIHN

SEQ ID NO: 7 - protein, 14 aa, artificial sequence
  substrate for Renin
DRVYIHPFHLVIHS

SEQ ID NO: 8 - protein, 10 aa, artificial sequence
  substrate for Renin
IHPFHLVIHT

SEQ ID NO: 9 - protein, 9 aa, artificial sequence
  substrate for Renin
RPFHLLVVY

SEQ ID NO: 10 - protein, 8 aa, artificial sequence
  substrate for Renin
PFHLLVYS

SEQ ID NO: 11 - protein, 16 aa, artificial sequence
  substrate for Anthrax Lethal Factor
MLARRKPVLPALTINP

SEQ ID NO: 12 - protein, 7 aa, artificial sequence
  substrate for Casapase-3
DEVDAPK

SEQ ID NO: 13 - protein, 13 aa, artificial sequence
  substrate for Cathepsin D
EDKPILFFRLGKE

SEQ ID NO: 14 - protein, 14 aa, artificial sequence
  substrate for Cathepsin D
DRVYIHPFHLLVYS

SEQ ID NO: 15 - protein, 5 aa, artificial sequence
  substrate for Cathepsin D
RGFFL

SEQ ID NO: 16 - protein, 5 aa, artificial sequence
  substrate for Cathepsin D
RGFFP

SEQ ID NO: 17 - protein, 11 aa, artificial sequence
  substrate for Cathepsin D
GKPILFFRLKR

SEQ ID NO: 18 - protein, 7 aa, artificial sequence
  substrate for Cathepsin D
FAAFFVL

SEQ ID NO: 19 - protein, 7 aa, artificial sequence
  substrate for Cathepsin D
FGHFFAF

SEQ ID NO: 20 - protein, 6 aa, artificial sequence
  substrate for Cathepsin D
FSFFAA

SEQ ID NO: 21 - protein, 7 aa, artificial sequence
  substrate for Cathepsin D
PTEFFRL

SEQ ID NO: 22 - protein, 8 aa, artificial sequence
  modified_base (6): Xaa is Abu
DEDEEXSK

SEQ ID NO: 23 - protein, 18 aa, artificial sequence
  substrate for Hepatitis C Virus (HCV) NS3 protease
EAGDDIVPCSMSYTWTGA

SEQ ID NO: 24 - protein, 12 aa, artificial sequence
  substrate for Human Cytomegalovirus (CMV) protease (Assemblin)
RGVVNASSRLAK

SEQ ID NO: 25 - protein, 11 aa, artificial sequence
  substrate for Human Cytomegalovirus (CMV) protease (Assemblin)
RGVVNASSRLA

SEQ ID NO: 26 - protein, 11 aa, artificial sequence
  substrate for Human T-Cell Leukemia Virus Type I (HTLV-I) protease
APQVLFVMHPL

SEQ ID NO: 27 - protein, 15 aa, artificial sequence
  substrate for SARS protease
VSVNSTLQSGLRKMA

SEQ ID NO: 28 - protein, 9 aa, artificial sequence
  substrate for Chymotrypsin
SEVNLDAEF

SEQ ID NO: 29 - protein, 6 aa, artificial sequence
  modified_base (4): Xaa is Nle
LSFXAL

SEQ ID NO: 30 - protein, 7 aa, artificial sequence
  substrate for Pepsin
FAAFFVL

SEQ ID NO: 31 - protein, 7 aa, artificial sequence
  substrate for Pepsin
FGHFFAF

SEQ ID NO: 32 - protein, 7 aa, artificial sequence
  substrate for Pepsin
PTEFFRL

SEQ ID NO: 33 - protein, 7 aa, artificial sequence
  modified_base (5): Phe (5) is NO2-Phe
PTEFFRL

SEQ ID NO: 34 - protein, 9 aa, artificial sequence
  substrate for Secretase
SEVNLDAEF

SEQ ID NO: 35 - protein, 4 aa, Artificial Sequence
  substrate for Coagulation Factor Xa, Kallikrein, and Coagulation Factor XIIa
IEGR

SEQ ID NO: 36 - protein, 4 aa, Artificial Sequence
  substrate for Caspase-3
DEVD

SEQ ID NO: 37 - protein, 4 aa, Artificial Sequence
  substrate for Caspase-3
DMQD

SEQ ID NO: 38 - protein, 4 aa, Artificial Sequence
  substrate for Caspase-3
DQMD

SEQ ID NO: 39 - protein, 4 aa, Artificial Sequence
  substrate for Chymotrypsin
AAPF

SEQ ID NO: 40 - protein, 4 aa, Artificial Sequence
  substrate for Chymotrypsin
AAPM

SEQ ID NO: 41 - protein, 4 aa, Artificial Sequence
  substrate for Chymotrypsin
AIPM

SEQ ID NO: 42 - protein, 4 aa, Artificial Sequence
  substrate for Trypsin
AAPR

SEQ ID NO: 43 - protein, 4 aa, Artificial Sequence
  substrate for Trypsin
AAPK

SEQ ID NO: 44 - protein, 4 aa, Artificial Sequence
  substrate for Pepsin
GGFF

SEQ ID NO: 45 - protein, 4 aa, Artificial Sequence
  substrate for Kallikrein
DPFR

SEQ ID NO: 46 - protein, 6 aa, Artificial Sequence
  substrate for Feline Immunodeficiency Virus (FFV) Protease
XXFXXX